(19) United States
(12) Patent Application Publication
(10) Pub. No.: US 2014/0288037 A1
(43) Pub. Date: Sep. 25, 2014

(54) NOVEL COMPOSITIONS AND METHODS FOR TREATING PROSTATE CANCER

(71) Applicants: David Scott Casebier, Carlisle, MA (US); Abdellah Sentissi, Dover, MA (US); Richard Christian Moreton, Waltham, MA (US); Mark Turnbull, Cambridge (CA)

(72) Inventors: David Scott Casebier, Carlisle, MA (US); Abdellah Sentissi, Dover, MA (US); Richard Christian Moreton, Waltham, MA (US); Mark Turnbull, Cambridge (CA)

(73) Assignee: Tokai Pharmaceuticals, Inc., Cambridge, MA (US)

(21) Appl. No.: 14/233,335

(22) PCT Filed: Jul. 18, 2012
(86) PCT No.: PCT/US2012/047253

§ 371 Date: Jun. 4, 2014

Related U.S. Application Data
(60) Provisional application No. 61/508,823, filed on Jul. 18, 2011.

(51) Int. Cl. C07J 43/00

(52) U.S. Cl. CPC C07J 43/003
USPC 514/176

(57) ABSTRACT

Described herein are compounds, methods of making such compounds, pharmaceutical compositions, and medicaments comprising such compounds, and methods of using such compounds to treat androgen receptor mediated diseases or conditions. In some embodiments, the solid matrix comprises a polymer. In some embodiments, the polymer is soluble in an aqueous solution. In particular embodiments, the aqueous solution is water. In other embodiments, the aqueous solution has a pH of 5.0 or greater.

### CROSS-REFERENCE

This application claims the benefit of U.S. Provisional Application No. 61/508,823, filed Jul. 18, 2011, which application is incorporated herein by reference.

### BACKGROUND

Cancer represents a significant burden on human health, accounting for an estimated 13% of all deaths each year. In particular, several common cancers and diseases are associated with androgen hormone signaling, such as, for example, prostate cancer, breast cancer, ovarian cancer, polycystic ovary disease. For example, prostate cancer is the most common cancer in men. The majority of prostate cancer deaths are due to the development of metastatic disease that is unresponsive to conventional androgen deprivation therapy. Androgen deprivation therapy has been the standard of care in subjects with prostate cancer since the 1940s. Despite androgen deprivation, most subjects ultimately experience disease progression. For many years this later phase of the disease was called “hormone insensitive prostate cancer” or “androgen independent prostate cancer.” It has since become clear that the prostate cancer that emerges after years of androgen deprivation therapy remains dependent upon androgen. The prostate cancer cells that have survived have gained the ability to import low levels of circulating androgens (expressed from adrenal glands), become much more sensitive to these low levels of testosterone, and actually synthesize testosterone within the prostate cancer cell itself. This stage of prostate cancer is now termed “castration resistant prostate cancer” or CRPC.

### SUMMARY OF THE INVENTION

In one aspect, the invention provides a solid dispersion composition comprising a compound of Formula I:

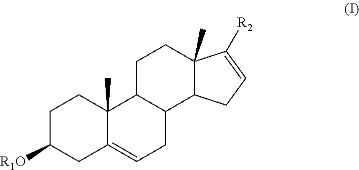

or a pharmaceutically acceptable salt, N-oxide, active metabolite, prodrug, or solvate thereof;
wherein: R₁ is H or acetyl; R₂ is pyridyl or benzimidazolyl; and a solid matrix; wherein said compound is dispersed in said solid matrix.

In some embodiments, the solid matrix comprises a polymer. In some embodiments, the polymer is soluble in an aqueous solution. In particular embodiments, the aqueous solution is water. In other embodiments, the aqueous solution has a pH of 5.0 or greater.

In some embodiments, the polymer is selected from the group consisting of 3,4-dimethyl-phenomethylcarbamate (MPMC), hydroxypropylmethylcelluolse acetate succinate (HPMCAS), hypromellose phthalate (HPMCP), Poloxamer 188, Poloxamer 407, poly(meth)acrylates (Eudragit), homopolymers of N-vinyl-2-pyrrolidone, povidone, copovidone (Plasdone), carboxymethylethylcellulose (CMEC), cellulose acetate phthalate (CAP), methacrylic copolymer LD (L30 D55), methacrylic copolymer S (S-100), aminoalkyl methacrylate copolymer E (gastric coating base), poly(vinyl acetal) diethylaminoacetate (AEA), polyvinylpyrrolidone (K-25, 50 30, 90; PVP), ethylcellulose (EC), methacrylic copolymer RS(RS 30D), polyvinyl alcohol (PVA), methylcellulose (MC), hydroxypropylcellulose (HPC), hydroxypropylmethylcellulose (HPMC), HPMC 2208 (Metolose 90SH), HPMC 2906 (Metolose 65SH), HPMC (Metolose 60SH), carboxymethylcellulose sodium (sodium cellulose glycolate), dextrin, pullulan, Acacia, tragacanth, sodium alginate, propylene glycol alginate, agar powder, gelatin, starch, processed starch, phospholipids, lecithin, glucomannan, block copolymers of ethylene oxide and propylene oxide (PEO/PPO), polyethyleneglycol (PEG) cellulose acetate trimellitate (CAT), hydroxypropyl methyl cellulose acetate trimellitate (HPMCAT), and carboxymethylcellulose acetate butyrate (CMCAB), or a random copolymer of N-vinyl-2-pyrrolidone and vinyl acetate. In particular embodiments, the polymer is HPMCAS, a poly(meth)acrylate, a homopolymer of N-vinyl-2-pyrrolidone, or a random copolymer of N-vinyl-2-pyrrolidone and vinyl acetate. In one embodiment, the polymer is HPMCAS.

In some embodiments, the composition further comprises one or more excipients. In some embodiments, the one or more excipients comprise one or more fillers, disintegrants, glidants, surfactants, recrystallization inhibitors, and/or lubricants. In some embodiments, the composition comprises one or more fillers. In particular embodiments, the one or more fillers comprise lactose monohydrate, microcrystalline cellulose, dicalcium phosphate, powdered cellulose, dextrates, or sodium bicarbonate. In one embodiment, the filler is lactose monohydrate. In some embodiments, the composition comprises one or more recrystallization inhibitors. In particular embodiments, the one or more recrystallization inhibitors comprise poloxamer 188, poloxamer 407, Povidone K-90, or hypromellose. In one embodiment, the one or more recrystallization inhibitors is poloxamer 188. In some embodiments, the composition comprises one or more disintegrants. In particular embodiments, the one or more disintegrants comprise croscarmellose sodium, sodium starch glycholate, or crospovidone. In one embodiment, the one or more disintegrants is crospovidone. In some embodiments, the composition comprises one or more surfactants. In one embodiment, the one or more surfactants is sodium lauryl sulfate. In some embodiments, the composition comprises one or more lubricants. In one embodiment, the one or more lubricants is magnesium stearate. In some embodiments, the composition comprises one or more glidants. In one embodiment, the one or more glidants is colloidal silicon dioxide.

In some embodiments, the compound accounts for 5-50% of said composition by weight. In particular embodiments, the compound accounts for 20-40% of the composition by weight.

In some embodiments, the solid matrix accounts for 5-80% of the composition by weight. In particular embodiments, the solid matrix accounts for 20-40% of the composition by weight.

In some embodiments, the weight ratio of the compound to the solid matrix is about 1:10-about 10:1. In particular embodiments, the weight ratio of the compound to the solid matrix is about 1:3-about 3:1. In one embodiment, the weight ratio of the compound to the solid matrix is about 1:1.

In some embodiments, the one or more excipients altogether account for 10-90% of the composition by weight. In particular embodiments, the one or more excipients altogether account for 15-60% of the composition by weight. In some embodiments, the weight ratio of excipient to compound is about 1:10-about 10:1. In particular embodiments, the weight ratio of excipient to compound is about 1:6-about 3:1. In more particular embodiments, the weight ratio of excipient to compound is about 1:2-about 2:1.

In some embodiments, the solid dispersion composition comprises about 15-45% of said compound by weight, 15-45% of said solid matrix, 5-40% of said one or more fillers, 2-25% of said one or more disintegrants, 0.5-15% of said one or more recrystallization inhibitors, 0.1-10% of said one or more glidants, and 0.1-2% of one or more lubricants. In other embodiments, the solid dispersion composition comprises about 15-40% of said compound, 15-40% of HPMCAS, 20-40% of lactose monohydrate, 5-25% of cropsovidone, 0.5-15% of poloxamer 188, 0.1-2% of colloidal silicon dioxide, and 0.1-2% of magnesium stearate. In other embodiments, the solid dispersion composition comprises about 20-40% said compound, 20-40% HPMCAS, 25-35% lactose monohydrate, 10-20% crospovidone, 2.5-7.5% polaxamer 188, 0.2-1% colloidal silicon dioxide, and 0.2-1% magnesium stearate. In other embodiments, the solid dispersion composition comprises 15-45% said compound, 15-45% HPMCAS, 20-40% microcrystalline cellulose, 5-25% crospovidone, 0.5-15% polaxamer 188, 0.1-10% colloidal silicon dioxide, and 0.1-2% magnesium stearate. In other embodiments, the solid dispersion composition comprises about 15-45% said compound, 15-45% copovidone, 20-40% microcrystalline cellulose, 5-25% crospovidone, 0.5-15% hypromellose NF, 0.1-10% colloidal silicon dioxide, and 0.1-2% magnesium stearate. In other embodiments, the solid dispersion composition comprises about 35-45% said compound, 35-45% HPMCAS, 5-15% dicalcium phosphate, 0.5-10% croscomellose sodium, 5-10% poloxomer 188, 0.1-2% colloidal silicon dioxide, and 0.1-2% magnesium stearate. In other embodiments, the solid dispersion composition comprises about 25-40% said compound, 25-40% copovidone, 15-30% sodium bicarbonate, 3-15% citric acid, 3-15% croscarmellose sodium, 2-10% hyrpomellose, 0.1-2% colloidal silicon disoxide, and 0.1-2% magnesium stearate.

In some embodiments, the solid dispersion composition is in the form of particles. In some embodiments, the particles have a median diameter of about 100 μm or less. In particular embodiments, the particles have a median diameter of about 50 μm or less. In yet more particular embodiments, the particles have a median diameter of 25 μm or less. In yet even more particular embodiments, the particles have a median diameter of about 20 μm or less. In some embodiments, the particles have a median diameter of about 10-20 μm. In some embodiments, 90% of the particles have a particle span distribution of about 17-19 μm.

In some embodiments, the particles have a bulk density of 0.14-0.45 g/ml. In particular embodiments, the particles have a bulk density of about 0.2-0.35 g/ml. In some embodiments, the particles have a tapped density of 0.3 g/ml or greater. In some embodiments, the composition comprises less than 4000 ppm of residual solvents.

In some embodiments, the composition is a powder. In other embodiments, the composition is a glassy, brittle solid material.

In another aspect, the invention provides a pharmaceutical composition comprising a compound of Formula:

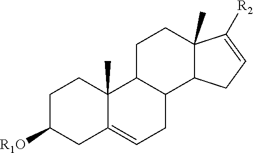

or a pharmaceutically acceptable salt, N-oxide, active metabolite, prodrug, or solvate thereof; wherein: R₁ is H or acetyl; R₂ is pyridyl or benzimidazolyl; the compound is substantially in a non-crystalline form, and the bioavailability of the compound when administered to a subject in a fasted state is substantially the same as the bioavailability of the drug when administered to the subject in a fed state. For example, R₁ is OH and R₂ is 1-benzimidazolyl. In another embodiment, R₁ is acetate and R₂ is 3-pyridyl.

In another aspect, the present invention provides compositions that are formulated such that the compound is amorphous. In some embodiments, any of the compositions of the present invention are formulated such that the compound is amorphous. In some embodiments, the compound is amorphous after storage of the composition at about 25-40° C./60-75% relative humidity (RH) for about 1 week or more. In some embodiments, the compound is amorphous after storage of the composition at about 2 weeks or more. In some embodiments, the compound is amorphous after storage of the composition at about one month or more.

In some embodiments, any of the compositions of the present invention are formulated to achieve at least about a 2-fold higher AUC or greater as compared to a composition comprising an equivalent amount of the compound in a crystalline form. In some embodiments, the composition is formulated to achieve at least about a 5-fold higher AUC or greater as compared to a composition comprising an equivalent amount of the compound in a crystalline form. In particular embodiments, the composition is formulated to achieve at least about a 10-fold higher AUC or greater as compared to a composition comprising an equivalent amount of the compound in a crystalline form. In some embodiments, the composition is formulated to achieve at least about a 2-fold higher CMax or greater as compared to a composition comprising an equivalent amount of the compound in a crystalline form, when each of the compositions is administered to a human subject. In some embodiments, the composition is formulated to achieve at least a 5-fold higher CMax or greater as compared to a composition comprising an equivalent amount of the compound in a crystalline form. In particular embodiments, the composition is formulated to achieve at least a 10-fold higher CMax or greater as compared to a composition comprising an equivalent amount of the compound in a crystalline form. In some embodiments, the composition and the equivalent amount of the compound substantially in crystalline form are tested by administration to a subject. In some embodiments, the subject is a mammal. In some embodiments, the subject is not a human. In other embodiments, the subject is a human. In some embodiments, the compositions are tested by administration to a subject in a fasted state. In some embodiments, the equivalent amount is about a 5-100 mg/kg dose. In some embodiments, the equivalent amount is about a 25-40 mg/kg dose. In one embodiment, the equivalent amount is about a 30 mg/kg dose. In some embodiments, the dose is a daily dose.

In some embodiments, any of the compositions of the present invention are formulated such that the dissolution rate of the composition in FaSSIF is 10-fold higher than a composition comprising the compound in substantially crystalline form. In some embodiments, the dissolution rate of the composition in FaSSIF is 50-fold higher than a composition comprising the compound in substantially crystalline form. In one embodiment, the dissolution rate of the composition in FaSSIF is 100-fold higher than a composition comprising the compound in substantially crystalline form.

In some embodiments, the bioavailability of the compound when administered to a subject in a fasted state is substantially the same as the bioavailability of the compound when administered to the subject in a fed state. In some embodiments, there is less than a 15% difference in the bioavailability of the compound when administered to a subject in a fasted state and the bioavailability of the drug when administered to the subject in a fed state. In some embodiments, the bioavailability is measured by comparing AUC and/or CMax of the compound in subjects in a fed vs. fasted state. In some embodiments, the difference between AUC and/or CMax between fed vs. fasted states in a subject is less than 30%, 25%, 20%, 15%, 10%, 5% or less.

In some embodiments, any of the compositions of the present invention are formulated such that the solubility of the compound after transition from pH 1-2 to pH 5-7 is no less than ⅓ the solubility of the compound at pH 1-2. In particular embodiments, the composition is formulated such that the solubility of the compound after transition from pH 1-2 to pH 5-7 is no less than ½ the solubility of the compound at pH 1-2. In more particular embodiments, the composition is formulated such that the solubility of the compound after transition from pH 1-2 to pH 5-7 is no less than ¾ the solubility of the compound at pH 1-2. In yet more particular embodiments, the composition is formulated such that the solubility of the compound after transition from pH 1-2 to pH 5-7 is no less than ⅘ the solubility of the compound at pH 1-2.

In some embodiments, the compound is a compound of Formula II:

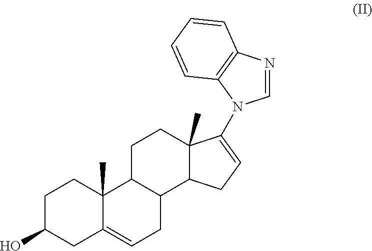 [SMILES: C[C@]12CC[C@H](O)CC1=CCC1C2CC[C@]2(C)C(n3cnc4ccccc43)=CCC12]

or a pharmaceutically acceptable salt, N-oxide, active metabolite, prodrug, or solvate thereof.

In some embodiments, the compound is a compound of Formula III:

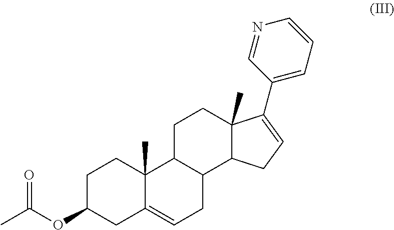 [SMILES: CC(=O)O[C@H]1CC[C@@]2(C)C(=CCC3C2CC[C@]2(C)C(c4cccnc4)=CCC32)C1]

or a pharmaceutically acceptable salt, N-oxide, active metabolite, prodrug, or solvate thereof.

In some embodiments, any of the compositions of the present invention are formulated as an oral dosage form, wherein the compound is present in a therapeutically effective amount for the treatment of cancer or other disease. In some embodiments, the oral dosage form is a solid oral dosage form. In particular embodiments, the solid oral dosage form is selected from the group consisting of a pill, tablet, capsule, pastille, lozenge, granule, or powder. In some embodiments, the tablet is a solid tablet, a buccal tablet, a sublingual tablet, an effervescent tablet, or chewable tablet. In some embodiments, the capsule is a hard-shelled capsule, a soft-gelled capsule, a roller compacted capsule, or a blended capsule.

In some embodiments, the invention provides a method of making a solid dispersion composition comprising a compound of Formula I:

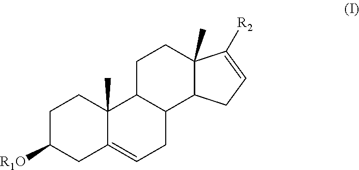

or a pharmaceutically acceptable salt, N-oxide, active metabolite, prodrug, or solvate thereof; wherein: R₁ is H or acetyl; R₂ is pyridyl or benzimidazolyl; the method comprising the steps of forming a solution comprising the compound, the solid matrix, and a solvent; and substantially removing the solvent, thereby resulting in the solid dispersion composition of the compound. For example, R₁ is OH and R₂ is 1-benzimidazolyl. In another embodiment, R₁ is acetate and R₂ is 3-pyridyl.

In some embodiments, the solvent comprises one or more organic compounds. In some embodiments, the one or more organic compounds are selected from the group consisting of dimethylformamide (DMF), acetone, methanol, ethanol, ethyl acetate, tetrahydrofuran, n-propanol, iso-propanol, butanol, methyl ethyl ketone, methyl iso-butyl ketone, propylacetate, acetonitrile, methylene chloride, toluene, 1,1,1-trichloroethane, dimethylacetamide, and dimethylsulfoxide. In particular embodiments, the solvent is selected from the group consisting of methanol, ethanol, ethyl acetate, acetone, tetrahydrofuran, 2:1 acetone:methanol, 2:1 methanol:tetrahydrofuran, 2:1 methanol:acetone, 6:1 DMF:water, 14:7:2:1 acetone:methanol: DMF: water, 4:1:1 methanol: water: acetone, 8:1 ethanol:water. In one embodiment, the solvent is 2:1 methanol:acetone.

In some embodiments, substantially removing the solvent comprises flash freezing the mixture and solvent followed by freeze-drying the mixture and solvent. In particular embodiments, the flash freezing the mixture and solvent followed by freeze drying is followed by drying the mixture in a centrifugal concentrator. In some embodiments, substantially removing the solvent comprises spray drying the mixture. In some embodiments, the spray drying comprises: atomizing the solution into a spray of droplets; and contacting the spray of droplets with a drying gas; wherein the contacting results in evaporation of the solvent, wherein the evaporation results in solid dispersion particles with substantially the same dimensions as the droplets. In some embodiments, the atomizing comprises delivering the solution through a spray nozzle. In particular embodiments, the atomizing comprises atomizing at an atomization pressure of about 0.8-1.4 bar. In one embodiment, the atomization pressure is about 1.2 bar. In some embodiments, the spray drying comprises delivering the solution through a spray-drying apparatus. In particular embodiments, the spray drying apparatus has an inlet temperature of about 80-110 degrees Celsius. In more particular embodiments, the spray drying apparatus has an inlet temperature of about 90 degrees Celsius. In some embodiments, the spray drying apparatus has an outlet temperature of about 50-65 degrees Celsius. In more particular embodiments, the spray drying apparatus has an outlet temperature of about 55 degrees Celsius. In some embodiments, the spray drying apparatus has a process gas flow of about 75-90 kg/hour. In more particular embodiments, the spray drying has a process gas flow of about 80 kg/hour. In one embodiment, the spray drying apparatus has an inlet temperature of about 90 degrees Celsius, an outlet temperature of about 55 degrees Celsius, an atomization pressure of about 1.2 bar, and a process gas flow of about 80 kg/hour. In some embodiments, removing the solvent additionally comprises a secondary drying process. In some embodiments, the method comprises blending the solid dispersion with one or more excipients described herein.

In some embodiments, the compound is a compound of Formula II:

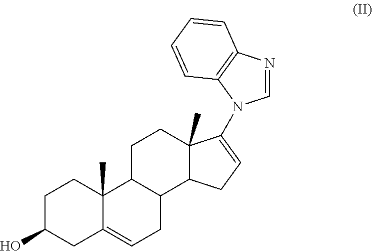 [SMILES: C[C@]12CC[C@H](O)CC1=CCC1C2CC[C@]2(C)C(n3cnc4ccccc43)=CCC12]

or a pharmaceutically acceptable salt, N-oxide, active metabolite, prodrug, or solvate thereof.

In other embodiments, the compound is a compound of Formula III:

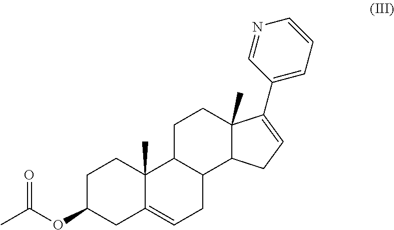 [SMILES: CC(=O)O[C@H]1CC[C@@]2(C)C(=CCC3C2CC[C@]2(C)C(c4cccnc4)=CCC32)C1]

or a pharmaceutically acceptable salt, N-oxide, active metabolite, prodrug, or solvate thereof.

In yet another aspect, the present invention provides a method of treating cancer in a subject in need thereof, comprising: obtaining a first sample from the subject; measuring a first amount of PSA in the first sample; administering a first cancer treatment comprising administration of a first substance for a duration of time; obtaining a second sample from the subject; measuring a second amount of PSA in the second sample; comparing the second amount to the first amount of PSA; and continuing the treatment if the second amount is decreased by 15% or more compared to the first amount or adjusting the treatment if the second amount is decreased by less than 15% compared to the first amount. In some embodiments, the first and second sample is a biological fluid. In particular embodiments, the biological fluid is blood plasma or serum. In some embodiments, the cancer treatment is a prostate cancer treatment. In some embodiments, the adjusting comprises discontinuing the first treatment. In some embodiments, the discontinuing is followed by starting a second treatment comprising administration of a second substance. In some embodiments, the first substance does not comprise a compound of Formula I, and wherein the second substance comprises a compound of Formula I. In some embodiments, treating comprises increasing the dosing regimen of the first treatment. In some embodiments, treating additionally comprises administration of a therapeutically effective amount of a second substance, wherein the second substance is distinct from the first substance. In some embodiments, the duration of time is about 1 week or more, 2 weeks or more, or one month or more. In some embodiments, treatment of the patient is continued if the patient's PSA level has decreased by at least about 25% after receiving the therapeutic compound for about 2 weeks. In some embodiments, treatment of the patient is adjusted if the patient's PSA level has decreased by less than about 20% after receiving the therapeutic compound for about 2 weeks.

In another aspect, the present invention provides a method for treating cancer or disease in a subject comprising administering to the subject a composition of any of the preceding claims. In some embodiments, the disease is polycystic ovarian disease. In some embodiments, the cancer is prostate cancer. In other embodiments, the cancer is not prostate cancer. In yet other embodiments, the cancer is breast cancer or ovarian cancer. In particular embodiments, the prostate cancer is castration resistant prostate cancer. In some embodiments, the patient has failed a treatment with ketoconazole. In some embodiments, the patient has failed a treatment with a lyase inhibitor. In some embodiments, the lyase inhibitor is Abiraterone. In some embodiments, the patient has failed a treatment with a second generation AR antagonist. In some embodiments, the second generation AR antagonist is MDV3100.

In some embodiments, the patient has failed a treatment with Lupron. In some embodiments, the patient has failed a chemotherapy treatment. In some embodiments, the composition is administered in multiple unit doses. In some embodiments, the unit dose is any oral dosage form described herein

In some embodiments, the invention contemplates a method for treating cancer in a patient comprising the step of administering a composition of Formula (I)

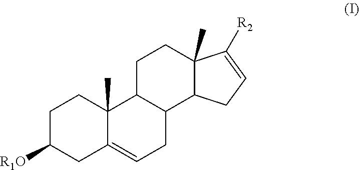

or a pharmaceutically acceptable salt, N-oxide, active metabolite, prodrug, or solvate thereof; wherein: R₁ is H or acetyl; R₂ is pyridyl or benzimidazolyl; wherein the composition is formulated to achieve an AUC of about 4750 h×ng/mL to about 32046 h×ng/mL. In some embodiments, the AUC is between about 4750 h×ng/mL to about 5925 h×ng/mL. In other embodiments, the AUC is between about 19354 h×ng/mL to about 32046 h×ng/mL. In yet other specific embodiments, the AUC is between about 14286 h×ng/mL to about 23714 h×ng/mL. For example, R₁ is OH and R₂ is 1-benzimidazolyl.

In some of these embodiments, 975 mg of compound I is administered in a single dose. In various embodiments, the composition is administered when the subject is in a fed state.

In some embodiments, the invention contemplates a method of treating a patient diagnosed with cancer comprising the steps of:

(1) determining the patient's PSA level;

(2) administering a therapeutic compound for about 2 weeks,

(3) determining the patient's PSA level after receiving the therapeutic compound for about 2 weeks; and

(4) continuing treatment of the patient with the therapeutic compound if the patient's PSA level has decreased by more than about 15% or discontinuing treatment of the patient with the therapeutic compound if the patient's PSA level has decreased by less than about 15%.

In some embodiments, the invention contemplates a pharmaceutical composition comprising

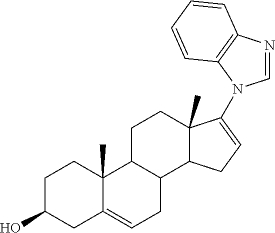 [SMILES: C[C@]12CC[C@H](O)CC1=CCC1C2CC[C@]2(C)C(n3cnc4ccccc43)=CCC12]

### Compound (I):

or a pharmaceutically acceptable salt, N-oxide, active metabolite, prodrug, or solvate thereof, wherein the compound is present in an amount of about 1950 mgs to about 3500 mgs.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a general workflow for preparing a spray dried dispersion formulation of TOK-001.

FIG. 2 depicts an XRPD plot of TOK-001:HPMCAS-SDD particles vs. micronized crystalline TOK-001 at T=0 after spray-drying (FIG. 2A), and after storage for one month at 40° C./75% relative humidity (FIG. 2B).

FIG. 3 depicts the impact of various recrystallization inhibitors on solubility of the TOK-001: HPMCAS SDD compositions in SGF and after transition from SGF to FaSSIF.

FIG. 4 depicts dissolution of various formulations of the TOK-001 compound as percent compound released into FaSSIF over time.

FIG. 5 depicts pharmacokinetic measurements of plasma TOK-001 concentrations in male Beagle dogs following oral administration of various formulations of the compound.

FIG. 6 depicts plasma concentrations of TOK-001 over time, comparing the TOK-001:HPMCAS SDD capsule formulation to the micronized crystalline PIC capsule formulation after administration to male Beagle dogs.

FIG. 7 depicts results from a human crossover trial, comparing plasma concentration of TOK-001the TOK-001:HPMCAS SDD capsule formulation to the micronized crystalline PIC capsule formulation

### DETAILED DESCRIPTION OF THE INVENTION

### Definitions

Adverse event: The term “adverse event” as used herein has its art understood meaning and refers to any untoward medical occurrence in a subject or clinical investigation subject administered a pharmaceutical product. An adverse event does not necessarily have to have a causal relationship with the treatment administered.

Adverse reaction: The term “adverse reaction” as used herein had its art understood meaning and refers to any noxious and unintended responses to a medicinal product related to any dose.

Combination Therapy: The term “combination therapy”, as used herein, refers to those situations in which two or more different pharmaceutical agents are administered in overlapping regimens so that the subject is simultaneously exposed to both agents.

Dosing Regimen: A “dosing regimen”, as that term is used herein, refers to a set of unit doses (typically more than one) that are administered individually separated by periods of time. The recommended set of doses (i.e., amounts, timing, route of administration, etc.) for a particular pharmaceutical agent constitutes its dosing regimen.

Initiation: As used herein, the term “initiation” when applied to a dosing regimen can be used to refer to a first administration of a pharmaceutical agent to a subject who has not previously received the pharmaceutical agent. Alternatively or additionally, the term “initiation” can be used to refer to administration of a particular unit dose of a pharmaceutical agent during therapy of a subject.

Pharmaceutical agent: As used herein, the phrase “pharmaceutical agent” refers to any agent that, when administered to a subject, has a therapeutic effect and/or elicits a desired biological and/or pharmacological effect.

Pharmaceutically acceptable ester: As used herein, the term “pharmaceutically acceptable ester” refers to esters which hydrolyze in vivo and include those that break down readily in the human body to leave the parent compound or a salt thereof.

Serious adverse event: The term “serious adverse event”, as used herein, has its art-understood meaning and refers to any untoward medical occurrence that at any dose, for example, results in death, is life threatening, requires insubject hospitalization (or prolongation of existing hospitalization), results in persistent or significant disability or incapacity (defined as a substantial disruption of a subject's ability to carry out normal life functions), etc. In some embodiments, a serious adverse event is a “serious adverse drug experience”, as that term is used by the United States Food and Drug Administration, for example as defined in 21 CFR §310.305(b), which says that a serious adverse event is any adverse drug experience occurring at any dose that results in any of the following outcomes: death, a life-threatening adverse drug experience, insubject hospitalization or prolongation of existing hospitalization, a persistent or significant disability/incapacity, or a congenital anomaly/birth defect. Important medical events that may not result in death, be life-threatening, or require hospitalization may be considered a serious adverse drug experience when, based upon appropriate medical judgment, they may jeopardize the subject or subject and may require medical or surgical intervention to prevent one of the outcomes listed in this definition. Examples of such medical events include allergic bronchospasm requiring intensive treatment in an emergency room or at home, blood dyscrasias or convulsions that do not result in insubject hospitalization, or the development of drug dependency or drug abuse.

Susceptible to: The term “susceptible to” is used herein to refer to an individual having higher risk (typically based on genetic predisposition, environmental factors, personal history, or combinations thereof) of developing a particular disease or disorder, or symptoms thereof, than is observed in the general population.

Therapeutically effective amount: The term “therapeutically effective amount” of a pharmaceutical agent or combination of agents is intended to refer to an amount of agent(s) which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). A therapeutically effective amount is commonly administered in a dosing regimen that may comprise multiple unit doses. For any particular pharmaceutical agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific pharmaceutical agent employed; the duration of the treatment; and like factors as is well known in the medical arts.

Treatment: As used herein, the term “treatment” (also “treat” or “treating”) refers to any administration of a pharmaceutical agent that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

Unit dose: The term “unit dose” or “dose”, as used herein, refers to a discrete administration of a pharmaceutical agent, typically in the context of a dosing regimen.

Definitions of standard chemistry terms may be found in reference works, including Carey and Sundberg “ADVANCED ORGANIC CHEMISTRY 4^{th} ED.” Vols. A (2000) and B (2001), Plenum Press, New York, hereby incorporated by reference in its entirety. Unless otherwise indicated, conventional methods of mass spectroscopy, NMR, HPLC, protein chemistry, biochemistry, recombinant DNA techniques and pharmacology, within the skill of the art are employed.

Solid dispersion: The term “solid dispersion”, as used herein, refers to composition comprising two different components, generally a solid matrix with a secondary substance (such as an active pharmaceutical ingredient) dispersed within.

Solid matrix: The term “solid matrix” refers to a solid phase in which molecules of a second substance (such as an active pharmaceutical ingredient) are embedded or dispersed within.

### Illustrative Biological Activity

### Androgen Receptor (AR)

Androgens bind to a specific receptor, the androgen receptor (AR), inside the cells of target tissues. The AR is expressed in numerous tissues of the body and is the receptor through which the physiological as well as the pathophysiological effects of endogenous androgen ligands, such as testosterone (T) and dihydrotestosterone (DHT), are expressed. Structurally, the AR is composed of three main functional domains: the ligand binding domain (LBD), the DNA-binding domain, and amino-terminal domain. A compound that binds to the AR and mimics the effects of an endogenous AR ligand is referred to as an AR agonist, whereas a compound that inhibits the effects of an endogenous AR ligand is termed an AR antagonist. Binding of androgen to the receptor activates it and causes it to bind to DNA binding sites adjacent to target genes. From there it interacts with coactivator proteins and basic transcription factors to regulate the expression of the gene. Thus, via its receptor, androgens cause changes in gene expression in cells. These changes ultimately have consequences on the metabolic output, differentiation or proliferation of the cell that are visible in the physiology of the target tissue. In the prostate, androgens stimulate the growth of prostate tissue and prostate cancer cells by binding to the AR that is present within the cytoplasm of androgen sensitive tissue.

Compounds which selectively modulate AR are of clinical importance in the treatment of or prevention of a variety of diseases, conditions, and cancers, including, but not limited to, prostate cancer, benign prostatic hyperplasia, hirsutism in women, alopecia, anorexia nervosa, breast cancer, acne, musculoskeletal conditions, such as bone disease, hematopoietic conditions, neuromuscular disease, rheumatological disease, cancer, AIDS, cachexia, for hormone replacement therapy (HRT), employed in male contraception, for male performance enhancement, for male reproductive conditions, and primary or secondary male hypogonadism.

### Castration Resistant Prostate Cancer

Agents that block the action (antiandrogens) of endogenous hormones (e.g., testosterone) are highly effective and routinely used for the treatment of prostate cancer (androgen ablation therapy). While initially effective at suppressing tumor growth, these androgen ablation therapies eventually fail in almost all subjects, leading to “castration resistant prostate cancer” (“CRPC”). Most, but not all, prostate cancer cells initially respond to androgen withdrawal therapy. However, with time, surviving populations of prostate cancer cells emerge because they have responded to the selective pressure created by androgen ablation therapy and are now refractory to it. Not only is the primary cancer refractory to available therapies, but cancer cells may also break away from the primary tumor and travel in the bloodstream, spreading the disease to distant sites (especially bone). Among other effects, this causes significant pain and further bone fragility.

It is contemplated that CRPC cells survive in an environment characterized by low levels of circulating androgens by amplifying at least three different pathways to enhance the response to the intracellular androgens that remain available. These include: (1) Up-regulation of the expression of the AR, which increases AR copy number and hence the sensitivity of the cells to low levels of circulating androgen induced by medical castration therapy; (2) Increase in the expression of enzymes involved in the importation of androgens that remain in cells after androgen deprivation therapy; (3) Increase in the expression of genes that regulate steroidogenesis, permitting the CRPC cells to synthesize their own androgens. A critical enzyme in the steroidogenic pathway is cytochrome C_{17α}-hydroxylase/C₁₇₋₂₀-lyase (CYP17), the enzyme that controls androgen production in the adrenals, testes, and prostate.

Described herein, in certain embodiments, are compounds, methods of making such compounds, pharmaceutical compositions and medicaments comprising such compounds, and methods of using such compounds to treat androgen receptor mediated diseases or conditions including, but not limited to, prostate cancer, benign prostatic hyperplasia, hirsutism in women, alopecia, anorexia nervosa, breast cancer, ovarian cancer, polycycstic ovary disease, acne, musculoskeletal conditions, such as bone disease, hematopoietic conditions, neuromuscular disease, rheumatological disease, cancer, AIDS, cachexia, for hormone replacement therapy (HRT), employed in male contraception, for male performance enhancement, for male reproductive conditions, and primary or secondary male hypogonadism. In some embodiments, the androgen receptor mediated disease or condition is prostate cancer. In some embodiments, the prostate cancer is castration resistant prostate cancer.

In some embodiments, the invention provides compounds, pharmaceutical compositions, and medicaments comprising such compounds, and methods of using such compounds that decrease androgen biosynthesis, decrease androgen receptor signaling and decrease androgen receptor sensitivity.

In one aspect, the compounds, pharmaceutical compositions and medicaments comprising such compounds, and methods of using such compounds decrease androgen biosynthesis. In some embodiments, the compounds disclosed herein inhibit the activity of enzymes that controls androgen production. In certain embodiments, the compounds disclosed herein inhibit the activity of cytochrome C_{17α}-hydroxylase/C₁₇₋₂₀-lyase (CYP17).

In one aspect, the compounds, pharmaceutical compositions and medicaments comprising such compounds, and methods of using such compounds decrease androgen receptor signaling. In some embodiments, the compounds disclosed herein bind to the AR and are a competitive inhibitor of testosterone binding.

In one aspect, the compounds, pharmaceutical compositions and medicaments comprising such compounds, and methods of using such compounds decrease androgen receptor sensitivity. In some embodiments, the compounds disclosed herein reduce the content of AR protein within the cell and diminish the ability of the cell to be sustained by low levels of androgenic growth signals.

### Exemplary Compounds

In one aspect, the invention provides novel compositions comprising a compound of Formula I

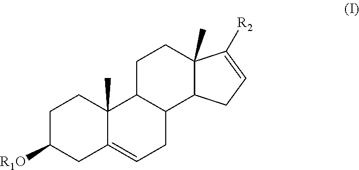

or a pharmaceutically acceptable salt, N-oxide, active metabolite, prodrug, or solvate thereof;
wherein R₁ is H or acetyl; R₂ is pyridyl or benzimidazolyl.

In some embodiments, the compound is a compound of Formula II:

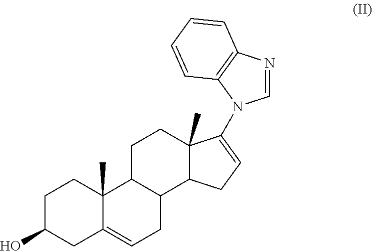 [SMILES: C[C@]12CC[C@H](O)CC1=CCC1C2CC[C@]2(C)C(n3cnc4ccccc43)=CCC12]

or a pharmaceutically acceptable salt, N-oxide, active metabolite, prodrug, or solvate thereof.

In other embodiments, the compound is a compound of Formula III:

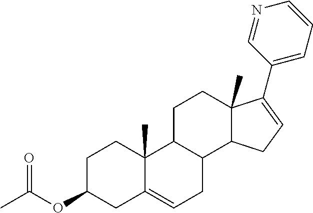 [SMILES: CC(=O)O[C@H]1CC[C@@]2(C)C(=CCC3C2CC[C@]2(C)C(c4cccnc4)=CCC32)C1]

or a pharmaceutically acceptable salt, N-oxide, active metabolite, prodrug, or solvate thereof;

The compounds of Formula I-III, pharmaceutically acceptable salts, pharmaceutically acceptable N-oxides, pharmaceutically active metabolites, pharmaceutically acceptable prodrugs, pharmaceutically acceptable polymorphs and pharmaceutically acceptable solvates thereof, modulate the activity of steroid hormone nuclear receptors and, as such, are useful for treating androgen receptor mediated diseases or conditions.

### Exemplary Synthesis of the Compounds

Compounds of Formula (II) (also described as Compound (I) or 3-β-Hydroxy17-(1H-benzimidazol-1-yl)androsta-5,16-diene) or TOK-001 or Galeterone) may be synthesized using standard synthetic techniques known to those of skill in the art or using methods known in the art in combination with methods described herein. Compounds of Formula (III) may be synthesized by similar methods. As one of skill in the art would understand, the solvents, temperatures and reaction conditions presented herein may vary according to the practice and knowledge of those of skill in the art.

The starting material used for the synthesis of the Compound (1) can be obtained from commercial sources, such as Aldrich Chemical Co. (Milwaukee, Wis.), Sigma Chemical Co. (St. Louis, Mo.), or the starting materials can be synthesized. The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference in their entirety). General methods for the preparation of compounds as disclosed herein may be derived from known reactions in the field, and the reactions may be modified by the use of appropriate reagents and conditions, as would be recognized by the skilled person, for the introduction of the various moieties found in the formulae as provided herein.

Compounds of Formula I-III can be prepared as a pharmaceutically acceptable acid addition salt (which is a type of a pharmaceutically acceptable salt) by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

Compounds of Formula I-III can be prepared as a prodrug. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they may be easier to administer than the parent drug. Prodrugs may, for instance, be bioavailable by oral administration whereas the parent is not. The prodrug may also have improved solubility in pharmaceutical compositions over the parent drug. An example, without limitation, of a prodrug would be a derivative of Formula (I-III), which is administered as a hydrophilic ester (the “prodrug”) to facilitate absorption in the gastrointestinal tract where improved water solubility is beneficial, but which then is metabolically hydrolyzed to a carboxylic acid and the active entity, Formula (I-III). A further example of a prodrug is a short peptide bonded to the hydroxyl group of Compound (I), wherein the peptide is metabolized to provide a compound of Formula I, II, or III.

Prodrugs may be designed as reversible drug derivatives for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Additionally, prodrug derivatives of compounds of Formula I-III can be prepared by methods known to those of ordinary skill in the art (e.g., for further details see Saulnier et al., (1994), Bioorganic and Medicinal Chemistry Letters, Vol. 4, p. 1985). Prodrug forms of the herein described compounds, wherein the prodrug is metabolized in vivo to produce a derivative as set forth herein are included within the scope of the claims. Indeed, some of the herein-described compounds may be a prodrug for another derivative or active compound.

Sites on the aromatic ring portion of compounds of Formula I-III can be susceptible to various metabolic reactions, therefore incorporation of appropriate substituents on the aromatic ring structures, for example, halogens, can reduce, minimize or eliminate this metabolic pathway.

Various methods of making compounds of Formula I-III are contemplated and the following descriptions are provided as non-limiting examples. In some embodiments, one or more of the following chemical reactions is performed in an inert atmosphere, for example, nitrogen or argon. In some embodiments, the temperature of the reaction is monitored. In some embodiments, the reaction is monitored by HPLC or TLC. In some embodiments, the pH of the reaction is monitored. In some embodiments, the temperature of the reaction is controlled. In some embodiments, the purity of the product is determined by HPLC. In some embodiments, the experiments are run on small scale, medium scale, large scale, analytical scale, or manufacturing scale. In some embodiments, the product is clarified by filtration through a pad comprising one or more of silica gel and celite.

In some embodiments, the synthesis is performed on large scale. In some embodiments, large scale comprises a scale of about 1 to about 10 kg. In some embodiments, the synthesis is performed on manufacturing scale. In some embodiments, manufacturing scale comprises a scale of greater than about 10 kg. In some embodiments, manufacturing scale comprises a scale of about 10 to about 1,000 kg. In some embodiments, manufacturing scale comprises a scale of about 10 to about 100 kg. In some embodiments, manufacturing scale comprises a scale of about 10 to about 50 kg. In some embodiments, manufacturing scale comprises a scale of about 33.4 kg.

In some embodiments, an experiment is performed on a smaller scale to gather information to be used to plan or perform synthesis on a manufacturing scale. In some embodiments, the results obtained on the smaller scales are expected to be reproducible on manufacturing scale. In some embodiments, the results obtained on smaller scales are not expected to be reproducible on manufacturing scale. In some embodiments, the yields obtained on manufacturing scale are greater than the yields obtained on smaller scales. In some embodiments, the yields obtained on manufacturing scale are lesser than the yields obtained on smaller scales.

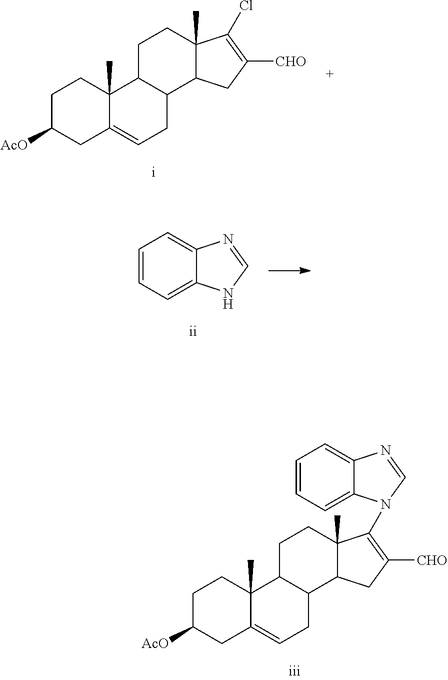 [SMILES: CC(=O)O[C@H]1CC[C@@]2(C)C(=CCC3C2CC[C@]2(C)C(Cl)=C(C=O)CC32)C1 ; c1ccc2[nH]cnc2c1 ; CC(=O)O[C@H]1CC[C@@]2(C)C(=CCC3C2CC[C@]2(C)C(n4cnc5ccccc54)=C(C=O)CC32)C1]

In one embodiment, a solution of a compound of Formula i in a solvent is prepared. A compound of Formula II is then contacted to the solution, and the resultant mixture is heated in the presence of a base for a period of time sufficient to provide a compound of Formula iii. In some embodiments, the period of time is about 1 hour, about 2 hours, about 4 hours, about 8 hours, about 12 hours, or about 24 hours. In some embodiments, the time is from about 1 hour to about 24 hours. In some embodiments, the base comprises lithium carbonate, sodium carbonate, potassium carbonate, sodium bicarbonate, a sodium phosphate, or a potassium phosphate. In some embodiments, the solvent comprises DMF. In some embodiments, the temperature is about 50° C., about 70° C., about 100° C., about 150° C., or a temperature effective to sustain reflux conditions. In some embodiments, the temperature is from about 50° C. to about 200° C. The compound of Formula iii can be isolated from the reaction mixture and purified by any method known to one of skill in the art. Such methods include, but are not limited to, pouring an aqueous mixture into the reaction mixture, thereby effecting the precipitation of compound iii as a solid. The isolated compound of Formula iii may optionally be purified by any method known to one of skill in the art. Such methods include, but are not limited to, trituration with water.

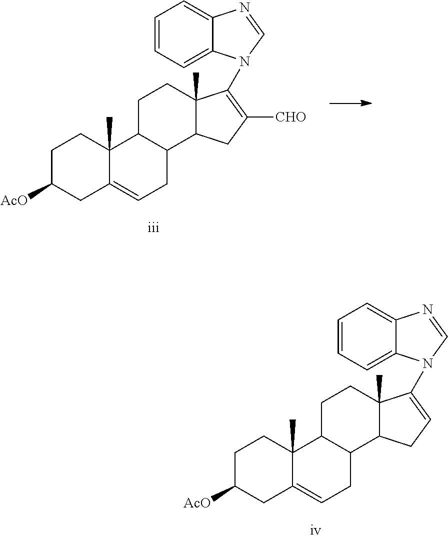 [SMILES: CC(=O)O[C@H]1CC[C@@]2(C)C(=CCC3C2CC[C@]2(C)C(n4cnc5ccccc54)=C(C=O)CC32)C1 ; CC(=O)O[C@H]1CC[C@@]2(C)C(=CCC3C2CC[C@]2(C)C(n4cnc5ccccc54)=CCC32)C1]

In one embodiment, a solution of a compound of Formula iii in a solvent is prepared, and the solution is contacted with a catalyst for a period of time sufficient to provide a compound of Formula iv. In some embodiments, the period of time is about 1 hour, about 2 hours, about 4 hours, about 8 hours, about 12 hours, or about 24 hours. In some embodiments, the time is from about 1 hour to about 24 hours. In some embodiments, the catalyst comprises palladium on carbon, platinum on carbon, a transition metal salt, or a transition metal complex. In some embodiments, the solvent comprises N-methylpyrrolidone. In some embodiments, the temperature is about 50° C., about 70° C., about 100° C., about 150° C., about 190° C., about 200° C., or a temperature effective to sustain reflux conditions. In some embodiments, the temperature is from about 50° C. to about 250° C. The compound of Formula iv can be isolated from the reaction mixture and purified by any method known to one of skill in the art. Such methods include, but are not limited to, in-line filtration. The isolated compound of Formula iv may optionally be purified by any method known to one of skill in the art.

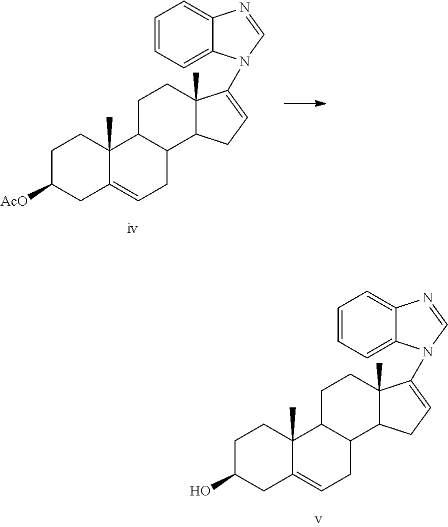 [SMILES: CC(=O)O[C@H]1CC[C@@]2(C)C(=CCC3C2CC[C@]2(C)C(n4cnc5ccccc54)=CCC32)C1 ; C[C@]12CC[C@H](O)CC1=CCC1C2CC[C@]2(C)C(n3cnc4ccccc43)=CCC12]

In one embodiment, a solution of a compound of Formula iv in a solvent is prepared, and the solution is contacted with a base for a period of time sufficient to provide a compound of Formula v (i.e., Compound (1)). In some embodiments, the period of time is about 1 hour, about 2 hours, about 4 hours, about 8 hours, about 12 hours, or about 24 hours. In some embodiments, the time is from about 1 hour to about 24 hours. In some embodiments, the base comprises lithium hydroxide, sodium hydroxide, potassium hydroxide, sodium methoxide, potassium methoxide, sodium ethoxide, potassium ethoxide, lithium carbonate, sodium carbonate, potassium carbonate, sodium bicarbonate, a sodium phosphate, or a potassium phosphate. In some embodiments, the solvent comprises water, methanol, ethanol, 2-propanol, t-butanol, or mixtures thereof. In some embodiments, the solvent comprises methanol and the base comprises sodium methoxide. In some embodiments, the temperature is about 35° C., about 50° C., about 70° C., about 100° C., or a temperature effective to sustain reflux conditions. In some embodiments, the temperature is from about 25° C. to about 100° C. The compound of Formula v can be isolated from the reaction mixture and purified by any method known to one of skill in the art. Such methods include, but are not limited to, extraction. The isolated compound of Formula v may optionally be purified by any method known to one of skill in the art. Such methods include, but are not limited to, trituration.

Pharmacokinetic Characteristics

Pharmacokinetic and pharmacodynamic data can be obtained by known techniques in the art. Due to the inherent variation in pharmacokinetic and pharmacodynamic parameters of drug metabolism in human subjects, appropriate pharmacokinetic and pharmacodynamic profile components describing a particular composition can vary. Typically, pharmacokinetic and pharmacodynamic profiles are based on the determination of the mean parameters of a group of subjects. The group of subjects includes any reasonable number of subjects suitable for determining a representative mean, for example, 5 subjects, 10 subjects, 16 subjects, 20 subjects, 25 subjects, 30 subjects, 35 subjects, or more. The mean is determined by calculating the average of all subject's measurements for each parameter measured.

The pharmacokinetic parameters can be any parameters suitable for describing the present composition. For example, the Cₘₐₓ can be not less than about 500 ng/ml; not less than about 550 ng/ml; not less than about 600 ng/ml; not less than about 700 ng/ml; not less than about 800 ng/ml; not less than about 880 ng/ml, not less than about 900 ng/ml; not less than about 100 ng/ml; not less than about 1250 ng/ml; not less than about 1500 ng/ml, not less than about 1700 ng/ml, or any other Cₘₐₓ appropriate for describing a pharmacokinetic profile of Compound (1). In some embodiments wherein the active metabolite is formed in vivo after administration of a drug to a subject; the Cₘₐₓ can be not less than about 500 pg/ml; not less than about 550 pg/ml; not less than about 600 pg/ml; not less than about 700 pg/ml; not less than about 800 pg/ml; not less than about 880 pg/ml, not less than about 900 pg/ml; not less than about 1000 pg/ml; not less than about 1250 pg/ml; not less than about 1500 pg/ml, not less than about 1700 pg/ml, or any other Cₘₐₓ appropriate for describing a pharmacokinetic profile of a compound formed in vivo after administration of Compound (I) to a subject.

The Tₘₐₓ can be, for example, not greater than about 0.5 hours, not greater than about 1.0 hours, not greater than about 1.5 hours, not greater than about 2.0 hours, not greater than about 2.5 hours, not greater than about 3.0 hours, not greater than 5.0 hours, or any other Tₘₐₓ appropriate for describing a pharmacokinetic profile of Compound (1).

The AUC_{(0-inf)} can be, for example, not less than about 590 ng·hr/mL, not less than about 1500 ng·hr/mL, not less than about 2000 ng·hr/mL, not less than about 3000 ng.times.hr/ml, not less than about 3500 ng·hr/mL, not less than about 4000 ng·hr/mL, not less than about 5000 ng·hr/mL, not less than about 6000 ng·hr/mL, not less than about 7000 ng·hr/mL, not less than about 8000 ng·hr/mL, not less than about 9000 ng·hr/mL, or any other AUC_{(0-inf)} appropriate for describing a pharmacokinetic profile of Compound (1). In some embodiments wherein an active metabolite is formed in vivo after administration of Compound (1) to a subject; the AUC_{(0-inf)} can be, for example, not less than about 590 pg·hr/mL, not less than about 1500 pg·hr/mL, not less than about 2000 pg·hr/mL, not less than about 3000 pg·hr/mL, not less than about 3500 pg·hr/mL, not less than about 4000 pg·hr/mL, not less than about 5000 pg·hr/mL, not less than about 6000 pg·hr/mL, not less than about 7000 pg·hr/mL, not less than about 8000 pg·hr/mL, not less than about 9000 pg·hr/mL, or any other AUC_{(0-inf)} appropriate for describing a pharmacokinetic profile of a compound formed in vivo after administration of Compound (1) to a subject.

The plasma concentration of Compound (1) about one hour after administration can be, for example, not less than about 140 ng/ml, not less than about 425 ng/ml, not less than about 550 ng/ml, not less than about 640 ng/ml, not less than about 720 ng/ml, not less than about 750 ng/ml, not less than about 800 ng/ml, not less than about 900 ng/ml, not less than about 1000 ng/ml, not less than about 1200 ng/ml, or any other plasma concentration of Compound (1).

The pharmacodynamic parameters can be any parameters suitable for describing the present composition. For example, the pharmacodynamic profile can exhibit decreases in AR protein or endogenous androgens for, by way of example only, at least about 2 hours, at least about 4 hours, at least about 8 hours, at least about 12 hours or at least about 24 hours. The pharmacodynamic profile can exhibit an inhibition of androgen synthesizing enzymes, including CYP17, for, by way of example only, at least about 2 hours, at least about 4 hours, at least about 8 hours, at least about 12 hours or at least about 24 hours. The pharmacodynamic profile can exhibit reduction of androgen signaling, for, by way of example only, at least about 2 hours, at least about 4 hours, at least about 8 hours, at least about 12 hours or at least about 24 hours.

In the current state of the art, compounds of Formula I are formulated as powder in capsule (PIC) formulations, in which the compound is in crystalline form, for oral administration. These formulations are associated with a number of limitations and potential safety profile issues. One existing concern regarding current formulations is the large variability in pharmacokinetics in subjects in a fed vs. fasted state. One exemplary current formulation exhibits widely divergent bioavailability in patients depending on their metabolic, e.g., fed vs. fasted, state. In particular, food has been reported to increase AUC 10-fold, and Cmax 17-fold, in patients. The large variability in bioavailability in subjects administered compositions comprising a compound of Formula I can lead to significant safety issues associated with unpredictable pharmacokinetics, particularly if taken with food. Due to these concerns, the formulation is indicated to only be taken in a fasted state (e.g., no food two hours before, or one hour after, oral administration). Therefore, in one aspect, the invention provides a composition comprising a compound of Formula I which is formulated to achieve similar pharmacokinetics when administered in a fed or a fasted state.

Comparative pharmacokinetics of the compound in fed vs. fasted states can be assessed using a number of methods that are well known in the art. In one example, pharmacokinetics can be indicated in vitro by measuring solubility of the compound in fasted or fed state simulated gastric fluid (FaSSGF vs. FeSSGF), and/or in fasted or fed state simulated intestinal fluid (FaSSIF vs. FeSSIF). In another example, pharmacokinetics can be indicated in vivo by conducting kinetic measurements of the amount of compound reaching the bloodstream after administration to live subjects that have been fed or fasted. In some embodiments, the live subjects used for comparative pharmacokinetics testing are animal subjects. In some embodiments, the subjects are mammals. In particular embodiments, the subjects are human subjects. In other embodiments, the subjects are non-human subjects such as, but not limited to canines, felines, non-human primates, rodents, birds, or reptiles. In some embodiments, the subjects are classified as being either in a fed or a fasted state. In some embodiments, a subject is classified as being in a fed state if the subject has ingested food from up to twelve hours prior to administration to four hours following administration. In particular embodiments, a subject is classified as being in a fed state if the subject has ingested food from up to six hours prior to administration to two hours following administration. In a particular embodiments, a subject is classified as being in a fed state if the subject has ingested food from up to two hours prior to administration to 1 hour following administration.

Non-limiting examples of kinetic measurements taken from live subjects include CMax (maximum concentration of the compound found in the blood stream following administration), AUC (area under the curve, calculated by integrating concentration measurements of the compound in the bloodstream over time), or TMax (time at which peak concentration of the compound is achieved following administration). In some embodiments, AUC_{inf} measurements are taken from subjects in a fed vs. fasted state. In particular embodiments, ratios are taken of the AUC_{inf}-fed/AUC_{inf}-fasted measurements. In more particular embodiments, the composition is deemed to achieve similar pharmacokinetics when administered in a fed or fasted state if the AUC_{inf}-fed/AUC_{inf}-fasted ratio is between 5-0.5, between 4.5-0.5, between 3.5-0.5, between 3-0.5, between 2.5-0.5, between 2-0.5, between 1.5-0.5, between 1.25-0.5, or between 1.20-0.75. In one embodiment, the composition is deemed to achieve similar pharmacokinetics when administered in a fed or fasted state if the AUC_{inf}-fed/AUC_{inf}-fasted ratio is between 1.25-0.75.

In some embodiments, CMax measurements are taken from subjects in a fed vs. fasted state. In particular embodiments, ratios are taken of the CMax-fed/CMax_fasted measurements. In more particular embodiments, the composition is deemed to achieve similar pharmacokinetics when administered in a fed or fasted state if the CMax-fed/CMax_fasted ratio is between 5-0.1, between 4.5-0.1, between 4-0.2, between 3.5-0.2, between 3-0.3, between 2.5-0.3, between 2-0.4, between 1.5-0.4, between 1.25-0.5, or between 1.1-0.65. In one embodiment, the composition is deemed to achieve similar pharmacokinetics when administered in a fed or fasted state if the CMax-fed/CMax_fasted ratio is between 1.1-0.65.

Another limitation of current compositions of the compound for oral administration is their limited bioavailability, which can necessitate larger doses. In another aspect, the present invention provides compositions with improved bioavailability compared to compositions comprising equivalent amounts of compound in crystalline form. Bioavailability can be indicated by pharmacokinetic parameters in in vitro models or in live subjects. Non-limiting examples of in vitro models and live subjects are described herein. In particular, in vitro models that provide useful indicators of bioavailability include, but are not limited to, dispersability in simulated gastric or intestinal fluid, dissolution in simulated gastric or intestinal fluid, or solubility in simulated gastric or intestinal fluid. Bioavailability can also be indicated by kinetic measurements taken from live subjects, examples of which are described herein. In some embodiments, improved bioavailability of the composition can be indicated by comparing the AUC of the composition compared to a micronized PIC formulation comprising an equivalent amount of compound in crystalline form. In some embodiments, the composition of the present invention is formulated to achieve an AUC that is at least 2-fold higher than the AUC of a composition comprising an equivalent amount of said compound in a crystalline form. In some embodiments, the composition of the present invention is formulated to achieve an AUC that is at least 5-fold higher than the AUC of a composition comprising an equivalent amount of said compound in a crystalline form. In some embodiments, the composition of the present invention is formulated to achieve an AUC that is at least 10-fold higher than the AUC of a composition comprising an equivalent amount of said compound in a crystalline form.

In some embodiments, improved bioavailability of the composition can be indicated by comparing the Cmaxof the composition compared to a micronized PIC formulation comprising an equivalent amount of compound in crystalline form. In some embodiments, the composition of the present invention is formulated to achieve a Cmaxthat is at least 2-fold higher than the Cmaxof a composition comprising an equivalent amount of said compound in a crystalline form. In some embodiments, the composition of the present invention is formulated to achieve a Cmaxthat is at least 5-fold higher than the Cmaxof a composition comprising an equivalent amount of said compound in a crystalline form. In some embodiments, the composition of the present invention is formulated to achieve a Cmaxthat is at least 10-fold higher than the Cmaxof a composition comprising an equivalent amount of said compound in a crystalline form.

In some embodiments, the invention provides a composition comprising a compound of Formula I that is formulated for rapid disintegration and/or dispersal in oral dosage form. Methods for determining disintegration and/or dispersal of pharmaceutical oral dosage forms are well known in the art. In some embodiments, the compositions are formulated for complete disintegration/dispersal in 15 minutes or less, 14 minutes or less, 13 minutes or less, 12 minutes or less, 11 minutes or less, 10 minutes or less, 8 minutes or less, 5 minutes or less, or 4 minutes or less.

Another limitation associated with currently available compositions of Formula I is their limited solubility in intestinal environments compared to gastric environments. Intestinal fluid typically has a pH of about 5-7, while gastric fluid can have pH ranging from 1-2. In particular, the switch from a low pH (1-2) to a high pH (5-7) environment can cause the compound to precipitate and crash out of solution, thus greatly limiting their solubility in high pH environments and subsequent bioavailability. Therefore, in some embodiments, the invention provides compositions comprising a compound of Formula I that are formulated such that the solubility of the compound is maintained after switching from an environment of pH 1-2 to an environment of pH 5-7. In some embodiments, the solubility of the compound after switching from pH 1-2 to pH 5-7 is no less than 1/10 the solubility of the compound at pH 1-2. In some embodiments, the solubility of the compound after switching from pH 1-2 to pH 5-7 is no less than ⅕ the solubility of the compound at pH 1-2. In some embodiments, the solubility of the compound after switching from pH 1-2 to pH 5-7 is no less than ⅓ the solubility of the compound at pH 1-2. In some embodiments, the solubility of the compound after switching from pH 1-2 to pH 5-7 is no less than ½ the solubility of the compound at pH 1-2. In some embodiments, the solubility of the compound after switching from pH 1-2 to pH 5-7 is no less than ¾ the solubility of the compound at pH 1-2.

In one aspect, the invention provides a composition comprising a compound of Formula I, wherein said compound is amorphous. By “amorphous”, it is meant that the majority of the compound in the composition is in an amorphous, that is, non-crystalline form. In some embodiments, about 50% or more, about 55% or more, about 60% or more, about 65% or more, about 70% or more, about 75% or more, about 80% or more, about 85% or more, about 90% or more, about 95% or more of the compound is in a non-crystalline state. In particular embodiments, about 80% or more of the compound is in a non-crystalline state. In yet more particular embodiments, about 90% or more of the compound is in a non-crystalline state. In one embodiment, 95% or more of the compound is in a non-crystalline state. Methods for determining whether a compound in a composition is amorphous are well known in the art, and include, but are not limited to Electron Microscopy, Polarized Light Microscopy, X-Ray Powder Diffraction (XPRD), Differential Scanning calorimetry (DSC), or other standard techniques.

In some embodiments, the compound in the composition remains amorphous for two weeks or more when stored under ambient conditions. The term “ambient conditions” generally refers to environments that are not artificially refrigerated, frozen, or heated. In some embodiments, the compound in the composition remains amorphous for two weeks or more when stored at room temperature. The term “room temperature” can be taken to mean temperatures between 10° C.-50° C., or between 15° C.-45° C., or between 20° C.-40° C. In other embodiments, the compound in the composition remains amorphous for two weeks or more when stored at relative humidity levels of 10-90%, 30-85%, 45-80%, or 60-75%. In some embodiments, the compound remains amorphous for up to one month, two months, three months, six months, one year, or more when stored under conditions described herein.

### Exemplary Pharmaceutical Compositions/Formulations

A pharmaceutical composition, as used herein, refers to a mixture of a compound of Formula I with other chemical components, such as carriers, stabilizers, diluents, dispersing agents, suspending agents, thickening agents, and/or excipients. The pharmaceutical composition facilitates administration of the compound to an organism. Pharmaceutical composition containing a compound of Formula I can be administered in therapeutically effective amounts as pharmaceutical compositions by any conventional form and route known in the art including, but not limited to: intravenous, oral, rectal, aerosol, parenteral, ophthalmic, pulmonary, transdermal, vaginal, otic, nasal, and topical administration.

One may administer the compound in a local rather than systemic manner, for example, via injection of the compound directly into an organ, often in a depot or sustained release formulation. Furthermore, one may administer pharmaceutical composition containing a compound of Formula I in a targeted drug delivery system, for example, in a liposome coated with organ-specific antibody. The liposomes will be targeted to and taken up selectively by the organ. In addition, the pharmaceutical composition containing a compound of Formula I may be provided in the form of a rapid release formulation, in the form of an extended release formulation, or in the form of an intermediate release formulation. In some embodiments, the extended release formulation releases the compound for over 1 hour, over 2 hours, over 3 hours, over 4 hours, over 6 hours, over 12 hours, over 24 hours, or more. In some embodiments, the extended release formulation releases the compound at a steady rate for over 1 hour, over 2 hours, over 3 hours, over 4 hours, over 6 hours, over 12 hours, over 24 hours, or more.

For oral administration, a compound of Formula I can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a subject to be treated. Generally, excipients such as fillers, disintegrants, glidants, surfactants, recrystallization inhibitors, lubricants, pigments, binders, flavoring agents, and so forth can be used for customary purposes and in typical amounts without affecting the properties of the compositions.

Non-limiting examples of fillers include lactose monohydrate, microcrystalline cellulose, mannitol, xylitol, calcium diphosphate, and starch.

Non-limiting examples of disintegrants include croscarmellose, sodium starch glycholate, crospovidone, sodium alginate, methyl cellulose, and carboxymethyl cellulose sodium.

Non-limiting examples of glidants include magnesium stearate, colloidal silicon dioxide, starch and talc.

Non-limiting examples of surfactants include sodium lauryl sulfate, sorbitan esters, poloxamers, PEG block copolymers, and polysorbates.

Non-limiting examples of recrystallization inhibitors include poloxamer 188, poloxamer 407, Povidone K-90, or hypromellose.

Non-limiting examples of lubricants include magnesium stearate and calcium stearate

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. In certain instances, a gelatin is alkaline processed. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner. Parental injections may involve for bolus injection or continuous infusion. The pharmaceutical composition of Compound (1) may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compositions described herein can be administered topically and can be formulated into a variety of topically administrable compositions, such as solutions, suspensions, lotions, gels, pastes, medicated sticks, balms, creams or ointments. Such pharmaceutical composition can contain solubilizers, stabilizers, tonicity enhancing agents, buffers and preservatives.

Formulations suitable for transdermal administration of compounds having the structure of Formula (1) may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of a compound of Formula I can be accomplished by means of iontophoretic patches and the like. Additionally, transdermal patches can provide controlled delivery of a compound of Formula I. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation, the compositions of the present invention may be in a form as an aerosol, a mist or a powder. Pharmaceutical compositions of Formula (I) are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

The compound of Formula I may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In practicing the methods of treatment or use provided herein, therapeutically effective amounts of a compound of Formula I provided herein are administered in a pharmaceutical composition to a mammal having a disease or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions comprising a compound of Formula (I) may be manufactured in a conventional manner, such as, by way of example only, by means of conventional mixing, dissolving, granulating, dragee-making, levigating, emulsifying, encapsulating, entrapping or compression processes.

The pharmaceutical compositions can include at least one pharmaceutically acceptable carrier, diluent or excipient and a compound of Formula (I) described herein as an active ingredient in free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

In some embodiments, the invention contemplates a pharmaceutical composition comprising Compound (1):

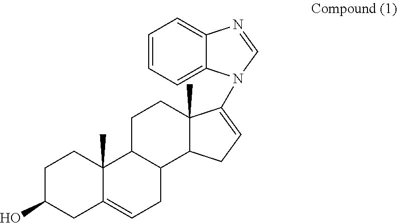 [SMILES: C[C@]12CC[C@H](O)CC1=CCC1C2CC[C@]2(C)C(n3cnc4ccccc43)=CCC12]

or a pharmaceutically acceptable salt, N-oxide, active metabolite, prodrug, or solvate thereof,
wherein the compound is present in an amount of about 1950 mgs to about 3500 mgs.

In some embodiments, the pharmaceutical composition comprises DMA, PEG 200, Cremophor EL, Solutol HS 15, NMP, Captisol, propylene glycol, 1N HCl, water or mixtures thereof.

In some embodiments, the pharmaceutical composition comprises DMA, PEG 200, Cremophor EL, Solutol HS 15, NMP, Captisol, propylene glycol or mixtures thereof.

In some embodiments, the pharmaceutical composition comprises DMA, PEG 200, Cremophor EL, water or mixtures thereof.

In some embodiments, the pharmaceutical composition comprises DMA, PEG 200, Solutol HS 15, water or mixtures thereof.

In some embodiments, the pharmaceutical composition comprises PEG 200, 1N HCl, water or mixtures thereof.

In some embodiments, the pharmaceutical composition comprises NMP, Captisol, water or mixtures thereof.

In some embodiments, the pharmaceutical composition comprises Solutol HS 15, NMP, propylene glycol, water or mixtures thereof.

In some embodiments, the pharmaceutical composition is a suspension dosage form.

In some embodiments, the suspension dosage form is a self-emulsifying drug delivery system.

In some embodiments, the self-emulsifying drug delivery system comprises propylene glycol, ethanol, castor oil, sesame oil, maisine 35-1, Capmul MCM, Labrasol, Labrafil M 2125CS, TPGS, Cremophor EL or a combination thereof.

In some embodiments, the self-emulsifying drug delivery system comprises propylene glycol, ethanol, castor oil, Labrafil M 2125CS, TPGS or a combination thereof.

In some embodiments, the self-emulsifying drug delivery system comprises ethanol, castor oil, maisine 35-1, TPGS, Cremophor EL or a combination thereof.

In some embodiments, the self-emulsifying drug delivery system comprises ethanol, sesame oil, Capmul MCM, Labrafil M 2125CS, TPGS, or a combination thereof.

In some embodiments, the self-emulsifying drug delivery system comprises ethanol, sesame oil, Labrasol, Cremophor EL or a combination thereof.

In some embodiments, the self-emulsifying drug delivery system comprises propylene glycol, castor oil, maisine 35-1, Labrasol, TPGS or a combination thereof.

In some embodiments, the self-emulsifying drug delivery system comprises ethanol, castor oil, Capmul MCM, Labrafil M 2125CS, TPGS, Cremophor EL or a combination thereof.

In some embodiments, the pharmaceutical composition comprises a lipid solid dispersion delivery system.

In some embodiments, the lipid solid dispersion delivery system comprises gelucire, a fat, a fatty acid, PEG, a block co-polymer, TPGS, a phospholipid, a non-ionic surfactant or a mixture thereof.

In some embodiments, the fat is a glyceride.

In some embodiments, the block co-polymer is a poloxamer.

In some embodiments, the non-ionic surfactant is a Tween.

In some embodiments, the lipid solid dispersion delivery system comprises gelucire 44/14.

In some embodiments, the lipid solid dispersion delivery system comprises PEG 1500.

In some embodiments, the lipid solid dispersion delivery system comprises TPGS.

In some embodiments, the lipid solid dispersion delivery system comprises Poloxamer 188.

In some embodiments, the lipid solid dispersion delivery system comprises gelucire 44/14, castor oil, Tween 20 or a mixture thereof.

In some embodiments, the lipid solid dispersion delivery system comprises gelucire 44/14, Poloxamer 188, castor oil or a mixture thereof.

In some embodiments, the lipid solid dispersion delivery system comprises gelucire 44/14, lecithin (soy) or a mixture thereof.

In some embodiments, the lipid solid dispersion delivery system comprises gelucire 44/14, cholic acid or a mixture thereof.

In some embodiments, the lipid solid dispersion delivery system comprises PEG 1500, TPGS or a mixture there of.

In some embodiments, the lipid solid dispersion delivery system comprises PEG 1500, Poloxamer 188 or a mixture thereof.

In some embodiments, the lipid solid dispersion delivery system comprises PEG 1500, castor oil, Tween 20 or a mixture thereof.

In some embodiments, the lipid solid dispersion delivery system comprises PEG 1500, Tween 20, lecithin (soy) or a mixture thereof.

In some embodiments, the lipid solid dispersion delivery system comprises PEG 1500, Tween 20, cholic acid or a mixture thereof.

In some embodiments, the pharmaceutical composition is a solid dispersion delivery system.

In some embodiments, the solid dispersion delivery system comprises hydroxypropyl methylcellulose (HPMC).

In some embodiments, the solid dispersion delivery system comprises hydroxypropyl methylcellulose phthalate (HPMCP).

In some embodiments, the solid dispersion delivery system comprises hydroxypropyl methylcellulose acetate succinate (HPMCAS).

In some embodiments, the solid dispersion delivery system comprises Poloxamer 188.

In some embodiments, the solid dispersion delivery system comprises Poloxamer 407.

In some embodiments, the solid dispersion delivery system comprises Povidone K-90.

In some embodiments, the pharmaceutical composition is a physical mixture.

A summary of types of pharmaceutical compositions may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference herein in its entirety.

Spray Dried Compositions and Methods

In some embodiments, the present invention provides solid dispersion compositions comprising a compound of Formula I:

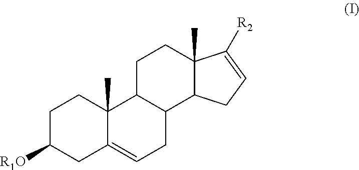

or a pharmaceutically acceptable salt, N-oxide, active metabolite, prodrug, or solvate thereof;
wherein R₁ is H or acetyl; R₂ is pyridyl or benzimidazolyl; and a solid matrix. In some embodiments, the compound of Formula I is dispersed in said solid matrix.

In some embodiments, the solid matrix is comprised of a polymer. In some embodiments, the polymer is a water soluble polymer. Non-limiting examples of water soluble polymers used in solid dispersions include hydroxypropyl methyl cellulose (HPMC), polyvinylpyrrolidone (PVPblock copolymers of ethylene oxide and propylene oxide ((K-25, 50 30, 90; PVP), hydroxypropyl cellulose (HPC), methyl cellulose (MC), and polyethyleneglycol (PEG). In other embodiments, the polymer is soluble in an aqeuous solution. In particular embodiments, the polymer is soluble in an aqueous solution which has a pH of 5.5 or greater. Non-limiting examples of polymers soluble in aqueous solutions of pH 5.5 or greater include sodium carboxymethylcellulose (NaCMC, sodium cellulose glycolate) and hydroxypropylmethyl cellulose acetate succinate (HPMCAS). Other non-limiting examples of polymers suitable for use in solid dispersions include, e.g., of 3,4-dimethyl-phenomethylcarbamate (MPMC), hypromellose phthalate (HPMCP), Poloxamer 188, Poloxamer 407, Povidone K-90, poly(meth)acrylates (Eudragit), homopolymers of N-vinyl-2-pyrrolidone, povidone, copovidone (Plasdone), carboxymethylethylcellulose (CMEC), cellulose acetate phthalate (CAP), methacrylic copolymer LD (L30 D55), methacrylic copolymer S (S-100), aminoalkyl methacrylate copolymer E (gastric coating base), poly(vinyl acetal) diethylaminoacetate (AEA), ethylcellulose (EC), methacrylic copolymer RS (RS 30D), polyvinyl alcohol (PVA), hydroxypropylmethylcellulose (HPMC), HPMC 2208 (Metolose 90SH), HPMC 2906 (Metolose 65SH), HPMC (Metolose 60SH), dextrin, pullulan, Acacia, tragacanth, sodium alginate, propylene glycol alginate, agar powder, gelatin, starch, processed starch, phospholipids, lecithin, glucomannan, polyethyleneglycol (PEG) cellulose acetate trimellitate (CAT), hydroxypropyl methyl cellulose acetate trimellitate (HPMCAT), and carboxymethylcellulose acetate butyrate (CMCAB).

In some embodiments, the solid dispersion of the compound in matrix can be prepared by forming a homogeneous solution or melt of the drug and polymer, followed by solidifying the mixture, resulting in a solid composition of the compound dispersed in the solid matrix. In some embodiments, preparation of the solid dispersion comprises forming a homogenous solution comprising the compound, the polymer, and a solvent, followed by solidifying the mixture by removal of the solvent. In some embodiments, the solvent is an organic solvent or a mixture of more than one organic solvent. Non-limiting examples of organic solvents include dimethylformamide (DMF), acetone, methanol, ethanol, ethyl acetate, tetrahydrofuran, n-propanol, iso-propanol, butanol, methyl ethyl ketone, methyl iso-butyl ketone, propylacetate, acetonitrile, methylene chloride, toluene, 1,1,1-trichloroethane, dimethylacetamide, and dimethylsulfoxide. In particular embodiments, the solvent is methanol, ethanol, ethyl acetate, acetone, tetrahydrofuran, 2:1 acetone: methanol, 2:1 methanol:tetrahydrofuran, 2:1 methanol:acetone, 6:1 DMF:water, 14:7:2:1 acetone: methanol: DMF: water, 4:1:1 methanol:water: acetone, 8:1 ethanol:water.

Methods for removing the solvent from the mixture are known in the art, and can include freeze-drying, vacuum drying, spray-drying, or combinations thereof.

In particular embodiments, the solvent is removed by spray-drying. The term “spray-drying” generally broadly refers to atomizing the solution into a spray of small droplets and rapidly removing solvent from the droplets using a spray-drying apparatus that facilitates rapid evaporation of solvent from the droplets. Spray-drying processes and spray-drying equipment are described generally in Perry's Chemical Engineers' Handbook, pages 20-54 to 20-57 (Sixth Edition 1984). Solvent evaporation can be facilitated by, e.g., maintaining the pressure in the spray-drying apparatus at a partial vacuum (for example, 0.01 to 0.50 atm), contacting the droplets with a warm drying gas, or a combination of these measures. In some embodiments, spray drying comprises contacting the spray of droplets with a drying gas.

In some embodiments, removal of the solvent by spray drying results in solid dispersion compositions in the form of particles. The particles can have a mean diameter of about 100 μm or less, about 95 μm or less, about 90 μm or less, about 85 μm or less, about 80 μm or less, about 75 μm or less, about 70 μm or less, about 65 μm or less, about 60 μm or less, about 55 μm or less, about 50 μm or less, about 45 μm or less, about 40 μm or less, about 35 μm or less, about 30 μm or less, about 25 μm or less, or about 20 μm or less. In some embodiments, the particles have a mean diameter of about 50-100 μm, about 30-75 μm, about 25-50 μm, about 20-30 μm, about 10-25 μm, or about 15-20 μm. Particle size can be measured using particle size measuring techniques known to those of skill in the art. Non-limiting examples of particle size measuring techniques include sedimentation field flow fractionation, photon correlation spectroscopy, laser diffraction or disk centrifugation. Another useful characteristic diameter of the droplets produced by an atomizer is D90, the droplet diameter corresponding to the diameter of droplets that make up 90% of the total liquid volume. In some embodiments, the particles of the composition have diameters spanning about 10-20 μm at D90, 15-20 μm at D90, or 17-19 μm at D90.

In some embodiments, spray-drying results in compositions in which the compound of Formula I is amorphous. Methods and characterization of amorphousness are described herein.

### Exemplary Methods of Administration and Treatment Methods

Compositions comprising a compound of Formula I-III can be used in the preparation of medicaments for the treatment of diseases or conditions in which steroid hormone nuclear receptor activity contributes to the pathology and/or symptoms of the disease. In addition, a method for treating any of the diseases or conditions described herein in a subject in need of such treatment, involves administration of pharmaceutical compositions containing at least one compound of Formula (1), or a pharmaceutically acceptable salt, pharmaceutically acceptable N-oxide, pharmaceutically active metabolite, pharmaceutically-acceptable prodrug, or pharmaceutically acceptable solvate thereof, in therapeutically-effective amounts to said subject.

The compositions containing the compound(s) described herein can be administered for prophylactic and/or therapeutic treatments. In therapeutic applications, the compositions are administered to a subject already suffering from a disease or condition, in an amount sufficient to cure or at least partially arrest the symptoms of the disease or condition, or to cure, heal, improve, or ameliorate the condition itself Amounts effective for this use will depend on the severity and course of the disease or condition, previous therapy, the subject's health status, weight, and response to the drugs, and the judgment of the treating physician.

Once improvement of the subject's conditions has occurred, a maintenance dose is administered if necessary. Subsequently, the dosage or the frequency of administration, or both, can be reduced, as a function of the symptoms, to a level at which the improved disease or condition is retained. Subjects can, however, require intermittent treatment on a long-term basis upon any recurrence of symptoms.

In certain instances, it may be appropriate to administer therapeutically effective amounts of at least one of the compounds described herein (or a pharmaceutically acceptable salts, pharmaceutically-acceptable N-oxides, pharmaceutically active metabolites, pharmaceutically-acceptable prodrugs, and pharmaceutically acceptable solvates thereof) in combination with another therapeutic agent. By way of example only, if one of the side effects experienced by a subject upon receiving one of the compounds herein is inflammation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the subject is enhanced). Or, by way of example only, the benefit of experienced by a subject may be increased by administering one of the compounds described herein with another therapeutic agent (which also includes a therapeutic regimen) that also has therapeutic benefit. In any case, regardless of the disease or condition being treated, the overall benefit experienced by the subject may simply be additive of the two therapeutic agents or the subject may experience a synergistic benefit. Where the compounds described herein are administered in conjunction with other therapies, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is one of the compounds described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents. Multiple therapeutic combinations are envisioned.

In addition, compounds of Formula I-III may also be used in combination with procedures that may provide additional or synergistic benefit to the subject. By way of example only, subjects are expected to find therapeutic and/or prophylactic benefit in the methods described herein, wherein pharmaceutical composition of Formula (I) and/or combinations with other therapeutics are combined with genetic testing to determine whether that individual is a carrier of a mutant gene that is known to be correlated with certain diseases or conditions.

Compounds of Formula I-III and combination therapies can be administered before, during or after the occurrence of a disease or condition, and the timing of administering the composition containing a compound can vary. Thus, for example, the compounds can be used as a prophylactic and can be administered continuously to subjects with a propensity to conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound is preferably administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, preferably about 1 month to about 3 years and in some embodiments from about 1 month to about 10 years. In other embodiments, the compound is administered once a day from 90 days to 2 years.

The pharmaceutical composition described herein may be in unit dosage forms suitable for single administration of precise dosages. In unit dosage form, the formulation is divided into unit doses containing appropriate quantities of one or more compounds. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The daily dosages appropriate for any of the compounds described herein are from about 0.03 to 60 mg/kg per body weight. An indicated daily dosage in a larger mammal, including, but not limited to, humans, is in the range from about 1 mg to about 4000 mg, conveniently administered in one or more doses, including, but not limited to, up to five times a day or in retard form. Suitable unit dosage forms for oral administration comprise from about 1 mg to about 4000 mg active ingredient. In some embodiments, a single dose of compounds of Formula (1) is within the range of about 50 mg to about 3500 mg. In some embodiments, a single dose of compounds of Formula (1) is about 90 mg, about 200 mg, about 250 mg, about 325 mg, about 500 mg, about 650 mg, about 975 mg, about 1300 mg, about 1625 mg, about 1950 mg, about 2600 mg or about 3250 mg. In some embodiments, an administration of compounds of Formula (1) of about 90 mg, about 325 mg, about 500 mg, about 650 mg, about 975 mg, about 1300 mg, about 1625 mg, about 1950 mg, about 2600 mg or about 3250 mg is given as multiple doses.

In some embodiments, the single dose of compounds of Formula (a) is between 90 to 3500 mgs and the compound is administered to a subject for between 90 days to two years.

Such dosages may be altered depending on a number of variables, not limited to the activity of the compound used, the disease or condition to be treated, the mode of administration, the requirements of the individual subject, the severity of the disease or condition being treated, and the judgment of the practitioner.

### Exemplary Methods of Providing Therapy

The present invention provides therapeutic strategies for the treatment of cancer or other disease in subjects. In some embodiments, the disease is polycystic ovarian disease. In some embodiments, the cancer in prostate cancer. In other embodiments, the cancer is breast cancer. In yet other embodiments, the cancer is ovarian cancer. In some embodiments, the subject is human. In other embodiments, the subject is not a human.

In particular embodiments, the present invention provides preparations and regimens for the use of a compound of Formula I in the treatment of prostate cancer. In some embodiments, the prostate cancer is castration resistance prostate cancer. In some embodiments, the prostate cancer is chemotherapy naïve prostate cancer.

In some embodiments, the present invention provides therapeutic regimens that involve oral administration of a compound of Formula I.

In some embodiments, the present invention provides therapeutic regimens that involve administration of multiple doses of a compound of Formula I. In some embodiments, different doses are spaced apart in time. In some embodiments, all doses contain the same amount of a compound of Formula I. In some embodiments, different doses contain different amounts of a compound of Formula I. In some embodiments, different doses that are separated in time are separated from one another by the same amount of time; in some embodiments, different doses that are separated in time are separated from one another by different amounts of time. In some embodiments, the present invention provides dosing regimens that include administration of a plurality of doses separated by a regular time interval (or intervals), followed by a rest period, optionally followed by a second plurality of doses separated by a regular time interval (or intervals).

In some embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168 or more doses of a compound of Formula I are administered. In some embodiments, at least 7, 14, 21, 28, 35, 42, 49, 56, 63, 70, 77, 84, 91, 98, 105, 112, 119, 126, 133, 140, 147, 154, 161, 168, or more doses of a compound of Formula I are administered.

In some embodiments, the invention contemplates a pharmaceutical composition comprising Compound (1):

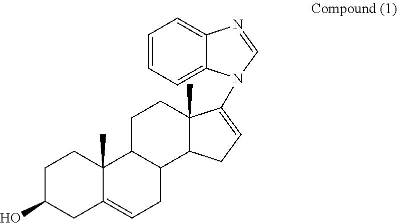 [SMILES: C[C@]12CC[C@H](O)CC1=CCC1C2CC[C@]2(C)C(n3cnc4ccccc43)=CCC12]

as a micronized crystalline powder.

In some embodiments, the invention contemplates a method for treating cancer in a patient comprising the step of administering a composition comprising Compound (1):

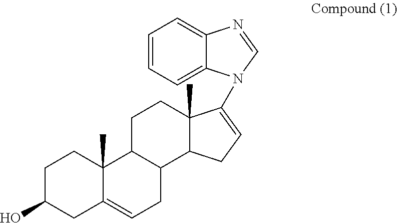 [SMILES: C[C@]12CC[C@H](O)CC1=CCC1C2CC[C@]2(C)C(n3cnc4ccccc43)=CCC12]

or a pharmaceutically acceptable salt, N-oxide, active metabolite, prodrug, or solvate thereof; wherein the composition is formulated to achieve an AUC of about 4750 h×ng/mL to about 32046 h×ng/mL. In some embodiments, the AUC is between about 4750 h×ng/mL to about 5925 h×ng/mL. In other embodiments, the AUC is between about 19354 h×ng/mL to about 32046 h×ng/mL. In yet other specific embodiments, the AUC is between about 14286 h×ng/mL to about 23714 h×ng/mL.

In some embodiments, the composition is about 1950 mg to about 3500 mg of Compound (1). In some embodiments the composition is less than 1950 mg of Compound (1).

In some embodiments, the patient has failed a treatment with ketoconazole.

In some embodiments, the patient has failed a treatment with a lyase inhibitor. In some embodiments, the lyase inhibitor is Abiraterone.

In some embodiments, the patient has failed a treatment with a second generation androgen receptor (AR) antagonist. In some embodiments, the second generation AR antagonist is MDV3100.

In some embodiments, the patient has failed a treatment with Lupron.

In some embodiments, the patient has failed a chemotherapy treatment.

In some embodiments, the invention contemplates a method of treating a patient diagnosed with cancer comprising the steps of:

(1) determining the patient's PSA level;

(2) administering a therapeutic compound for about 2 weeks,

(3) determining the patient's PSA level after receiving the therapeutic compound for about 2 weeks; and

(4) continuing treatment of the patient with the therapeutic compound if the patient's PSA level has decreased by more than about 15% or discontinuing treatment of the patient with the therapeutic compound if the patient's PSA level has decreased by less than about 15%.

In some embodiments, the treatment of the patient is continued if the patient's PSA level has decreased by at least about 25% after receiving the therapeutic compound for about 2 weeks.

In some embodiments, Compound (1) is present in an amount effective to treat an androgen receptor mediated disease or condition after administration to a subject.

In some embodiments, the androgen receptor mediated disease or condition is selected from the group consisting of prostate cancer, benign prostatic hyperplasia, hirsutism, alopecia, anorexia nervosa, breast cancer, and male hypergonadism.

In some embodiments, the androgen receptor mediated disease or condition is prostate cancer.

In some embodiments, the prostate cancer is castration resistant prostate cancer.

In some embodiments, Compound (1) is present in an amount effective to inhibit androgen biosynthesis, inhibit androgen receptor signaling and decrease androgen receptor sensitivity after administration to a subject.

In some embodiments, the compound inhibits androgen receptor signaling or decreases androgen receptor sensitivity.

In some embodiments, the androgen biosynthesis inhibition comprises inhibiting the activity of cytochrome C_{17α}-hydroxylase/C17, 20-lyase (CYP17).

In some embodiments, the androgen receptor signaling inhibition comprises competitive inhibition of testosterone binding.

In some embodiments, the decrease in androgen receptor sensitivity comprises a reduction of the content of androgen receptor protein within the cell, and a diminished ability of the cell to be sustained by low levels of androgenic growth signals.

In some embodiments, the composition is formulated for administration to a subject parenterally, intravenously, intramuscularly, intradermally, subcutaneously, intraperitoneally, orally, buccally, sublingually, mucosally, rectally, transcutaneously, transdermally, ocularly, or by inhalation.

In some embodiments, the composition is formulated for administration to a subject as a tablet, a capsule, a cream, a lotion, an oil, an ointment, a gel, a paste, a powder, a suspension, an emulsion, or a solution.

In some embodiments, the composition is formulated for administration to a subject as a capsule.

The pharmaceutical composition of any of the preceding claims, wherein the composition is formulated for administration to a subject as a tablet.

In some embodiments, the capsule comprises Compound (1) as a powder.

In some embodiments, the powder is micronized.

In some embodiments, the composition comprises about 50 mg to about 500 mg of Compound (1).

In some embodiments, the composition comprises about 100 mg to about 350 mg of Compound (1).

In some embodiments, the composition comprises about 90 mg of Compound (1).

In some embodiments, the composition comprises about 325 mg of Compound (1).

In some embodiments, the composition is formulated for administration to a subject, one, two, three, four, five, six, seven, eight, nine, or ten times per day.

In some embodiments, the composition is formulated to be administered to a subject for the treatment of prostate cancer.

In some embodiments, the composition is formulated to be administered to a subject for the treatment of castration resistant prostate cancer.

In some embodiments, the composition further comprises one or more pharmaceutically acceptable excipients.

In some embodiments, the pharmaceutically acceptable excipient comprises a filler, a disintegrant, a lubricant, a surfactant, a glidant, a binder, a sugar, a starch, a varnish, or a wax.

In some embodiments, compound (1) is a pharmaceutically acceptable salt, N-oxide, active metabolite, prodrug, crystalline polymorph, or solvate.

In some embodiments, the solvate comprises a cumene solvate or a hydrate.

In some embodiments, the invention contemplates a method comprising contacting dimethylformamide, potassium carbonate, a compound of the formula:

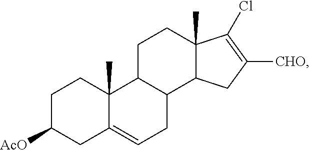 [SMILES: CC(=O)O[C@H]1CC[C@@]2(C)C(=CCC3C2CC[C@]2(C)C(Cl)=C(C=O)CC32)C1]

or analogue thereof, and a compound of the formula:

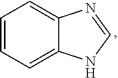

to make a compound of the formula:

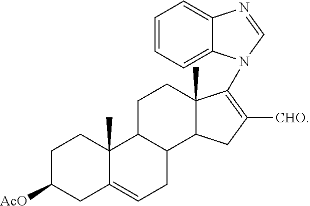 [SMILES: CC(=O)O[C@H]1CC[C@@]2(C)C(=CCC3C2CC[C@]2(C)C(n4cnc5ccccc54)=C(C=O)CC32)C1]

In some embodiments, the method further comprises contacting a compound of the formula:

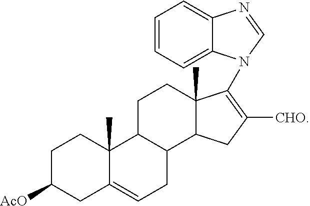 [SMILES: CC(=O)O[C@H]1CC[C@@]2(C)C(=CCC3C2CC[C@]2(C)C(n4cnc5ccccc54)=C(C=O)CC32)C1]

with 10% palladium on charcoal in N-methylpyrrolidone to produce a compound of the formula:

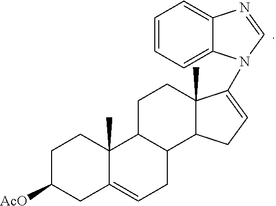 [SMILES: CC(=O)O[C@H]1CC[C@@]2(C)C(=CCC3C2CC[C@]2(C)C(n4cnc5ccccc54)=CCC32)C1]

In some embodiments, the method further comprises contacting a compound of the formula:

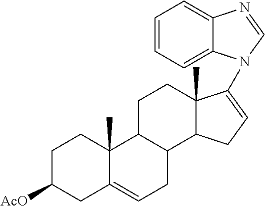 [SMILES: CC(=O)O[C@H]1CC[C@@]2(C)C(=CCC3C2CC[C@]2(C)C(n4cnc5ccccc54)=CCC32)C1]

with methanolic sodium methoxide to produce a compound of formula (I):

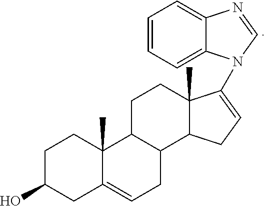 [SMILES: C[C@]12CC[C@H](O)CC1=CCC1C2CC[C@]2(C)C(n3cnc4ccccc43)=CCC12]

In some embodiments, the method is performed at a large scale or a manufacturing scale. In some embodiments, large scale is a scale of about 1 to about 10 kg. In some embodiments, manufacturing scale is a scale of greater than about 10 kg. In some embodiments, manufacturing scale is a scale of about 10 to about 1,000 kg. In some embodiments, manufacturing scale is a scale of about 10 to about 100 kg. In some embodiments, manufacturing scale is a scale of about 10 to about 50 kg. In some embodiments, manufacturing scale is a scale of about 33.4 kg.

### ILLUSTRATIVE EXAMPLES

The following examples provide illustrative methods for making and testing the effectiveness and safety of compositions comprising a compound of Formula I-III. These examples are provided for illustrative purposes only and not to limit the scope of the claims provided herein. All of the methods disclosed and claimed herein can be made and executed without undue experimentation in light of the present disclosure.

It will be apparent to those of skill in the art that variations may be applied to the methods and in the steps or in the sequence of steps of the method described herein without departing from the concept, spirit and scope of the claims. All such similar substitutes and modifications apparent to those skilled in the art are deemed to be within the spirit, scope and concept of the appended claims.

### Example 1

### Synthesis of Compounds of Formula (1)

### Example 1A

### Synthesis of 3-β-Acetoxy-17-(1H-benzimidazol-1-yl)-16-formylandrosta-5,16-diene

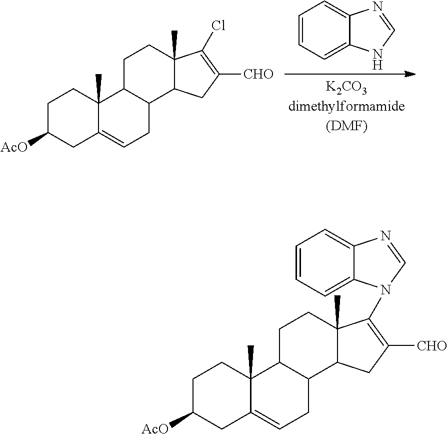 [SMILES: CC(=O)O[C@H]1CC[C@@]2(C)C(=CCC3C2CC[C@]2(C)C(Cl)=C(C=O)CC32)C1 ; c1ccc2[nH]cnc2c1 ; CC(=O)O[C@H]1CC[C@@]2(C)C(=CCC3C2CC[C@]2(C)C(n4cnc5ccccc54)=C(C=O)CC32)C1]

33.4 kg of 3-β-acetoxy-17-chloro-16-formylandrosta-5,16-diene was mixed with benzimidazole and potassium carbonate in dimethylformamide (DMF) and heated until the reaction was complete as determined by the amount of starting material remaining. After the reaction was complete, the reaction mixture was cooled and mixed with cooled water to quench the reaction. The solid was isolated from the quenched reaction mixture and washed sequentially with a mixture of DMF and water, water, dilute aqueous hydrochloric acid, water, dilute aqueous sodium hydrogen carbonate, and water. The intermediate product, 3-β-Acetoxy17-(1H-benzimidazol-1-yl)-16-formylandrosta-5,16-diene, was subsequently dried.

### Example 1B

### Synthesis and Purification of 3-β-Acetoxy-17-(1H-benzimidazol-1-yl)androsta-5,16-diene

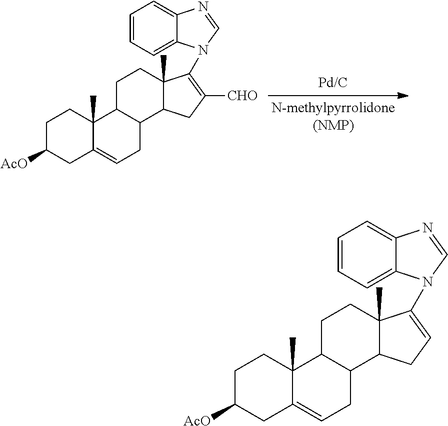 [SMILES: CC(=O)O[C@H]1CC[C@@]2(C)C(=CCC3C2CC[C@]2(C)C(n4cnc5ccccc54)=C(C=O)CC32)C1 ; CC(=O)O[C@H]1CC[C@@]2(C)C(=CCC3C2CC[C@]2(C)C(n4cnc5ccccc54)=CCC32)C1]

3-β-Acetoxy-17-(1H-benzimidazol-1-yl)-16-formylandrosta-5,16-diene was mixed with about 10% palladium on carbon (Pd/C) in N-methylpyrrolidone (NMP) and heated until the reaction was complete as determined by the 3-β-Acetoxy-17-(1H-benzimidazol-1-yl)-16-formylandrosta-5,16-diene/3-β-Acetoxy-17-(1H-benzimidazol-1-yl)androsta-5,16-diene ratio in the reaction mixture. After the reaction was complete, the reaction mixture was cooled. Magnesium sulfate was added, and the resulting mixture was filtered. Water was added to the filtrate and the resulting mixture was stirred. The solid, crude 3-β-Acetoxy17-(1H-benzimidazol-1-yl)androsta-5,16-diene was isolated from the water/NMP mixture, washed with a mixture of water and methanol, dried, and packaged.

The crude 3-β-Acetoxy-17-(1H-benzimidazol-1-yl)androsta-5,16-diene was dissolved in ethyl acetate and clarified. The volume of this mixture was reduced by vacuum distillation. The resulting mixture was cooled, and the solid was isolated, washed with cold ethyl acetate, and dried under vacuum. In some embodiments, a sample was subjected to an in-process test to determine impurity levels. If the impurity levels were not acceptable, a recrystallization process was repeated.

### Example 1C

### Synthesis and Purification of 343-Hydroxy-17-(1H-benzimidazol-1-yl)androsta-5,16-diene

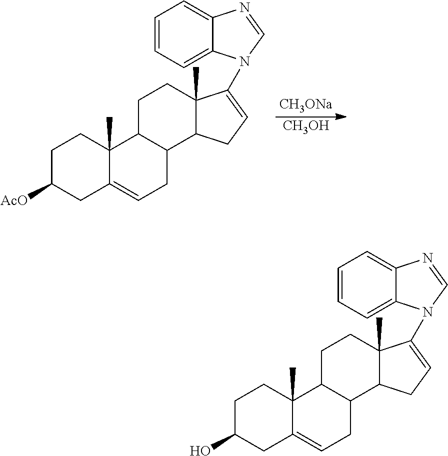

3-β-Acetoxy-17-(1H-benzimidazol-1-yl)androsta-5,16-diene was mixed with sodium methoxide in methanol and heated until the reaction was complete as determined by the amount of 3-β-Acetoxy-17-(1H-benzimidazol-1-yl)androsta-5,16-diene remaining. After the reaction was complete, the reaction mixture was cooled and mixed with water to quench the reaction. The resulting slurry was stirred and cooled further. The solid, crude 3-β-Hydroxy 17-(1H-benzimidazol-1-yl)androsta-5,16-diene was isolated from the quenched reaction mixture and washed with a mixture of methanol and water and then with water until the wash liquid was neutral, dried, and packaged.

The crude 3-β-Hydroxy-17-(1H-benzimidazol-1-yl)androsta-5,16-diene was dissolved in a mixture of methanol and ethyl acetate and clarified. The product was transferred from the methanol/ethyl acetate solution to ethyl acetate alone by solvent exchange. The resulting mixture was cooled, and the solid was isolated, washed with cold ethyl acetate, and dried under vacuum. In some embodiments, a sample was subjected to an in-process test to determine impurity levels. If the impurity levels were not acceptable, a recrystallization process was repeated.

### Example 2

### Pharmaceutical Compositions

### Example 2A

### Oral Composition

To prepare a pharmaceutical composition for oral delivery, a compound of Formula (1) was micronized to have a bulk density of about 0.20 g/mL and a tap density of about 0.31 g/mL. 90 mg of micronized compound was pack-filled into size “3” capsules suitable for oral administration.

### Example 2B

### Oral Composition

To prepare a pharmaceutical composition for oral delivery, a compound of Formula (1) was micronized to have a bulk density of about 0.20 g/mL and a tap density of about 0.31 ng/mL. 325 mg of micronized compound was pack-filled into size “00” capsules suitable for oral administration.

### Example 2C

### Oral Composition

To prepare a pharmaceutical composition for oral delivery, 90 mg of a compound of Formula (1) is mixed with 200 mg of lactose and 1% magnesium stearate. The mixture is blended and directly compressed into a tablet suitable for oral administration.

### Example 2D

### Parenteral Composition

To prepare a parenteral pharmaceutical composition suitable for administration by injection, 100 mg of a water-soluble salt of a compound of Formula (1) is dissolved in DMSO and then mixed with 10 mL of 0.9% sterile saline. The mixture is incorporated into a dosage unit form suitable for administration by injection.

### Example 2E

### Standard Vehicles

### Preparation of SEDDS/Equilibrium Solubility

Approximately 20 mg of compound (1) was added to each of six microcentrifuge tubes, and 1 mL of the appropriate vehicle (Table 1) was added to each to create a suspension. The capped tubes were mixed on a laboratory rotator at ambient temperature. At approximately 2, 24, and 48 hours after sample preparation the tubes were removed from the rotator and centrifuged to separate the solid phase from the solution. An aliquot of the supernatant was withdrawn from each sample and diluted as necessary for HPLC analysis to determine the solution concentration of compound (1), which was quantitated relative to external standards. The results are presented in Table 2.

TABLE 1
Standard vehicles.

| | Vehicle Components (% w/w) | | | | | | | | |
|---|---|---|---|---|---|---|---|---|---|
| Vehicle | | | Cremophor | Solutol Hs | | | Propylene | | Water |
| # | DMA | PEG 200 | EL | 15 | NMP | Captisol | glycol | 1N HCI | (WFI) |
| 1 | 10 | 30 | 5 | | | | | | 55 |
| 2 | 10 | 15 | 3 | | | | | | 72 |
| 3 | 5 | 10 | | | | | | | 85 |
| 4 | | 30 | | | | | | 10 | 60 |
| 5 | | | | | 5 | 20 | | | 75 |
| 6 | | | | 3 | 10 | | 15 | | 72 |

TABLE 2
Solubility in standard vehicles.

| | [Compound (1)], | [Compound (1)], | [Compound (1)], |
|---|---|---|---|
| Vehicle # | mg/mL, 2 h | mg/mL, 24 h | mg/mL, 48 h |
| 1 | <0.4 | 0.373 | 0.367 |
| 2 | <0.4 | 0.227 | 0.214 |
| 3 | <0.4 | <0.02 | <0.004 |
| 4 | 0.609 | 0.741 | 0.788 |
| 5 | 2.17 | 2.57 | 2.58 |
| 6 | <0.4 | 0.209 | 0.210 |

### In Vitro Assessment

The performance of the vehicle #5 formulation was evaluated in vitro by dilution into fasted- and fed-state simulated gastric and intestinal fluids immediately after the 72 hour solubility time point.

The supernatant of the formulation was diluted into FaSSGF and FeSSGF maintained at ambient temperature. The dilution ratio was 1:10 (v/v) for the fasted-state experiment and 1:20 for the fed state experiment. For 15 minutes, each dilution was agitated to ensure consistent movement of the liquid. The resultant mixtures were monitored visually for appearance of precipitate, and there appeared to be none. After 15 minutes, the samples were centrifuged to pellet undissolved solids, if any. The resultant solutions were assayed for pH and for compound (1) concentration by HPLC.

A portion of the mixture diluted into fasted-state simulated gastric fluid (FaSSGF) and fed-state simulated gastric fluid (FeSSGF) was mixed by vortex agitation to re-suspend any undissolved compound, if present, for a subsequent dilution into the corresponding fasted- or fed-state simulated intestinal fluid (FaSSIF or FeSSIF) maintained at ambient temperature. The dilution ratio was 1:10 (v/v) for both fasted and fed states. The mixtures were agitated to ensure consistent movement of the liquid and monitored visually for appearance of precipitate for 15 minutes. Precipitate was apparent in the FeSSIF dilution. The resultant solutions were assayed for pH and for compound (1) concentration by HPLC. The results of the in vitro evaluation of the formulation are summarized in Table 3.

TABLE 3
In vitro evaluation of standard vehicle #5.

| | [Compound (1)] in | % Recovery from | |
|---|---|---|---|
| Medium | medium, mg/mL | theoretical | pH |
| FaSSGF | 0.233 | 97.9% | 1.76 |
| FaSSIF | 0.017 | 76.5% | 6.37 |
| FeSSGF | 0.120 | 96.0% | 4.96 |
| FeSSIF¹ | <0.004 | <35.3% | 5.70 |

¹Concentration of HPLC sample below LOQ. Percent recovery therefore is presented as less than the recovery of a sample with a concentration at the LOQ.

### Example 2F

### Self-Emulsifying Drug Delivery Systems (SEDDS)

### Preparation of SEDDS/Equilibrium Solubility

Approximately 20 mg of compound (1) was added to each of six microcentrifuge tubes, and 1 ml, of the appropriate vehicle (Table 4) was added to each to create a suspension. The capped tubes were mixed on a laboratory rotator at ambient temperature. If all of compound (1) dissolved, more was added to maintain saturation. At approximately 2, 24, 48, and 72 hours after sample preparation the tubes were removed from the rotator and centrifuged to separate the solid phase from the solution. An aliquot of the supernatant was withdrawn from each sample and diluted with n-octanol as necessary for UV spectrophotometry analysis to determine the solution concentration of the compound (1), which was quantitated relative to external standards prepared in n-octanol. The final time point was taken immediately prior to in vitro evaluation of the formulations. The linearity of the response to the standards prepared in n-octanol was confirmed. The results of the solubility study are presented in Table 5.

TABLE 4
Self-emulsifying drug delivery systems.

| | % Vehicle component (w/w) | | | | | | | | | |
|---|---|---|---|---|---|---|---|---|---|---|
| Vehicle | Propylene | | | | | Capmul | | Labrafil M | | Cremphor |
| # | Gycol | Ethanol | Castor Oil | Sesame Oil | Maisline 35-1 | MCM | Labrasol | 2125CS | TPGS | EL |
| 7 | 10 | 10 | 35 | | | | | 30 | 15 | |
| 8 | | 10 | 30 | | 30 | | | | 10 | 20 |
| 9 | | 10 | | 30 | | 30 | | 20 | 10 | |
| 10 | | 10 | | 30 | | | 40 | | | 20 |
| 11 | 20 | | 30 | | 20 | | 15 | | 15 | |
| 12 | | | 15 | 35 | | 15 | | 15 | 10 | 10 |

TABLE 5
Solubility in self-emulsifying drug delivery systems.

| | | [Compound | [Compound | [Compound |
|---|---|---|---|---|
| | [Compound (1)], | (1)], | (1)], | (1)], |
| Vehicle # | mg/mL, 2 h | mg/mL, 24 h | mg/mL, 48 h | mg/mL, 72 h |
| 7 | 24.4 | 15.3 | 18.9 | 27.4 |
| 8 | 16.6 | 6.34 | 15.3 | 29.4 |
| 9 | 26.3 | 24.1 | 35.7 | 33.4 |
| 10 | 8.42 | 4.63 | 19.1 | 25.9 |
| 11 | 11.9 | 6.87 | 20.4 | 18.6 |
| 12 | 19.2 | 27.2 | 34.5 | 34.7 |

### In Vitro Assessment

The performance of the six SEDDS formulations was evaluated in vitro by dilution into fasted- and fed-state simulated gastric and intestinal fluids immediately after the 72 hour solubility time point.

The supernatant of each formulation was diluted into FaSSGF and FeSSGF maintained at ambient temperature. The dilution ratio was 1:10 (v/v) for the fasted-state experiment and 1:20 for the fed state experiment. For 15 minutes, each dilution was agitated to ensure consistent movement of the liquid.

A portion of the mixture diluted into FaSSGF and FeSSGF was mixed by vortex agitation to re-suspend any undissolved compound, if present, for a subsequent dilution into the corresponding fasted- or fed-state simulated intestinal fluid maintained at ambient temperature. The dilution ratio was 1:10 (v/v) for both fasted and fed states. The mixtures were agitated to ensure consistent movement of the liquid for 15 minutes.

Upon removal from the rotator, all samples were centrifuged and a portion of the clear solution was added to a fixed volume of n-octanol. The samples were rotated overnight at ambient temperature to extract compound (1) into the n-octanol layer. The samples were centrifuged, and the resultant n-octanol solutions were analyzed by UV spectrophotometry.

The results of the in vitro evaluation of the formulations are presented in Table 6. (Note: due to the emulsifying nature of the formulations, recoveries of greater than 100% are likely due to transfer of undissolved material).

TABLE 6
In vitro evaluation of self-emulsifying drug delivery systems.

| | | [Compound (1)] in | % Recovery from | |
|---|---|---|---|---|
| Vehicle # | Medium | medium, mg/mL | theoretical | pH |
| 7 | FaSSGF | 0.759 | 30% | 1.66 |
| | FaSSIF | 0.063 | 28% | 6.38 |
| | FeSSGF | 0.208 | 16% | 4.95 |
| | FeSSIF | 0.157 | 132% | 5.72 |
| 8 | FaSSGF | 1.16 | 44% | 1.66 |
| | FaSSIF | 0.130 | 54% | 6.37 |
| | FeSSGF | 0.509 | 36% | 4.95 |
| | FeSSIF | 0.179 | 141% | 5.72 |
| 9 | FaSSGF | 0.135 | 4% | 1.66 |
| | FaSSIF | 0.123 | 45% | 6.40 |
| | FeSSGF | 0.334 | 21% | 4.93 |
| | FeSSIF | 0.169 | 117% | 5.70 |
| 10 | FaSSGF | 1.44 | 61% | 1.66 |
| | FaSSIF | 0.146 | 68% | 6.40 |
| | FeSSGF | 0.230 | 19% | 4.95 |
| | FeSSIF | 0.111 | 99% | 5.71 |
| 11 | FaSSGF | 0.505 | 30% | 1.65 |
| | FaSSIF | 0.063 | 41% | 6.38 |
| | FeSSGF | 0.158 | 18% | 4.94 |
| | FeSSIF | 0.112 | 139% | 5.69 |
| 12 | FaSSGF | 1.26 | 40% | 1.67 |
| | FaSSIF | 0.163 | 57% | 6.39 |
| | FeSSGF | 0.490 | 30% | 4.94 |
| | FeSSIF | 0.167 | 111% | 5.70 |

### Example 2G

### Lipid Solid Dispersions

### Preparation of Lipid Solid Dispersions

Approximately 400 mg of compound (1) was added to each of 13 5-mL glass vials, each containing a magnetic stir bar. Approximately 1.6 g of molten vehicle (Table 4) (melted in an 80° C. oven) was added to the vial; vehicles were vortex mixed thoroughly before dispensing. The vehicles solidified rapidly after addition to the compound (1). The resultant mixtures were heated to approximately 60° C. in a water bath to melt the vehicles and stirred on a stir plate to produce uniform suspensions containing approximately 20% (w/w) of compound (1). The compound did not completely dissolve in any of the vehicles. The stir bars were removed and the mixtures cooled in a water bath to speed solidification and reduce settling of the compound.

TABLE 7
Lipid solid dispersions.

| | % Vehicle component (w/w) | | | | | | | |
|---|---|---|---|---|---|---|---|---|
| Vehicle | Gelucire | | | Poloxamer | | | Lecithin | Cholic |
| # | 44/14 | PEG 1500 | TPGS | 188 | Castor oil | Tween 20 | (soy) | acid |
| 13 | 100 | | | | | | | |
| 14 | | 100 | | | | | | |
| 15 | | | 100 | | | | | |
| 16 | | | | 100 | | | | |
| 17 | 75 | | | | 15 | 10 | | |
| 18 | 50 | | | 40 | 10 | | | |
| 19 | 90 | | | | | | 10 | |
| 20 | 95 | | | | | | | 5 |
| 21 | | 50 | 50 | | | | | |
| 22 | | 50 | | 50 | | | | |
| 23 | | 75 | | | 15 | 10 | | |
| 24 | | 85 | | | | 5 | 10 | |
| 25 | | 90 | | | | 5 | | 5 |

### In Vitro Assessment

The performance of 13 lipid solid dispersion formulations was evaluated in vitro by dilution into fasted- and fed-state simulated gastric and intestinal fluids.

For the fasted-state experiment, a quantity of approximately 100 mg of sample was dispensed into a microcentrifuge tube, and a volume of approximately 1 mL of FaSSGF was added (a 1:10 (w/v) dilution). The samples were manually dispersed using a spatula before adding medium. Brittle vehicles such as #16 and #22 formed powders easily, whereas soft, waxy vehicles such as #13 could not be ground but were instead smeared in the tube to increase the surface area exposed to the fluid.

The samples were briefly mixed by vortex agitation and placed on a rotator at ambient temperature for 15 minutes. The samples were again mixed by vortex agitation, and a 100 [EL aliquot of each suspension was diluted into 1 mL FaSSIF. These samples were briefly mixed by vortex agitation and placed on the rotator for 15 minutes at ambient temperature. The pH values of the SGF and SIF samples were measured.

The fed-state experiment was performed in the same way, except a volume of approximately 1 mL of FeSSGF was added to a quantity of approximately 50 mg lipid solid dispersion (a 1:20 (w/v) dilution). The FeSSGF sample was diluted into FeSSIF as described above.

Upon completion of each dilution experiment (incubation in simulated fluids for 15 minutes with agitation), the samples were centrifuged and a portion of the clear solution was added to a fixed volume of n-octanol. The samples were rotated overnight at ambient temperature to extract compound (1) into the n-octanol. The samples were centrifuged, and the resultant n-octanol solutions were analyzed by UV spectrophotometry. Note that most samples appeared as clear, uniform solutions rather than two layers; the FeSSIF samples each contained a small pellet after centrifugation.

The results of the in vitro evaluation of the formulations are presented in Table 8. The recovery in SGF and SIF from each formulation was determined from the theoretical concentration, in mg/mL, in each SGF sample based on the mass of the lipid solid dispersion, the percentage of compound (1) in that formulation, and the volume of medium added.

TABLE 8
In vitro evaluation of lipid solid dispersions.

| | | [Compound (1)] | | |
|---|---|---|---|---|
| Lipid solid | | in medium, | % Recovery from | |
| dispersion # | Medium | mg/mL | theoretical | pH |
| 13 | FaSSGF | 0.979 | 4.9% | 1.61 |
| | FaSSIF | 0.048 | 2.7% | 6.36 |
| | FeSSGF | 0.153 | 1.5% | 4.97 |
| | FeSSIF | 0.119 | 13.2% | 5.00 |
| 14 | FaSSGF | 0.243 | 1.2% | 1.65 |
| | FaSSIF | 0.165 | 9.2% | 6.36 |
| | FeSSGF | 0.353 | 3.6% | 4.97 |
| | FeSSIF | 0.173 | 19.2% | 5.72 |
| 15 | FaSSGF | 2.86 | 14.5% | 1.68 |
| | FaSSIF | 0.165 | 9.2% | 6.37 |
| | FeSSGF | 0.605 | 6.2% | 4.96 |
| | FeSSIF | 0.196 | 21.9% | 5.69 |
| 16 | FaSSGF | 1.31 | 7.1% | 1.64 |
| | FaSSIF | 0.017 | 1.0% | 6.35 |
| | FeSSGF | 0.004 | 0.04% | 4.99 |
| | FeSSIF | 0.199 | 23.8% | 5.72 |
| 17 | FaSSGF | 1.30 | 6.6% | 1.67 |
| | FaSSIF | 0.055 | 3.1% | 6.35 |
| | FeSSGF | 0.195 | 2.0% | 4.97 |
| | FeSSIF | 0.133 | 14.9% | 5.72 |
| 18 | FaSSGF | 0.574 | 2.8% | 1.69 |
| | FaSSIF | 0.041 | 2.2% | 6.35 |
| | FeSSGF | 0.046 | 0.5% | 4.98 |
| | FeSSIF | 0.157 | 17.2% | 5.71 |
| 19 | FaSSGF | 0.594 | 3.0% | 1.70 |
| | FaSSIF | 0.043 | 2.4% | 6.36 |
| | FeSSGF | 0.182 | 1.8% | 4.98 |
| | FeSSIF | 0.111 | 12.4% | 5.72 |
| 20 | FaSSGF | 1.75 | 8.7% | 1.68 |
| | FaSSIF | 0.050 | 2.7% | 6.33 |
| | FeSSGF | 0.121 | 1.2% | 4.96 |
| | FeSSIF | 0.119 | 13.0% | 5.71 |
| 21 | FaSSGF | 1.61 | 8.1% | 1.70 |
| | FaSSIF | 0.103 | 5.7% | 6.33 |
| | FeSSGF | 0.345 | 3.5% | 4.98 |
| | FeSSIF | 0.173 | 19.2% | 5.72 |
| 22 | FaSSGF | 0.352 | 1.8% | 1.71 |
| | FaSSIF | 0.021 | 1.2% | 6.35 |
| | FeSSGF | 0.074 | 0.8% | 5.04 |
| | FeSSIF | 0.152 | 17.0% | 5.70 |
| 23 | FaSSGF | 0.608 | 3.1% | 1.69 |
| | FaSSIF | 0.033 | 1.8% | 6.36 |
| | FeSSGF | 0.011 | 0.1% | 5.00 |
| | FeSSIF | 0.113 | 12.7% | 5.69 |
| 24 | FaSSGF | 0.041 | 0.2% | 1.68 |
| | FaSSIF | 0.022 | 1.2% | 6.34 |
| | FeSSGF | 0.078 | 0.8% | 5.02 |
| | FeSSIF | 0.223 | 24.8% | 5.69 |
| 25 | FaSSGF | 0.110 | 0.6% | 1.69 |
| | FaSSIF | 0.024 | 1.4% | 6.31 |
| | FeSSGF | 0.207 | 2.1% | 5.00 |
| | FeSSIF | 0.146 | 16.5% | 5.70 |

### Example 2H

### Solid Dispersions

### Preparation of Solid Dispersions—Method 1 Flash Freezing and Freeze Drying

Approximately 100 mg of compound (1) and 400 mg of the specified additive (Table 9) were dispensed into each of six tubes. The mixtures were dissolved in the solvent systems described in Table 9. Aliquots of 5-10 mL of each solution were distributed into 10 mL lyophilization vials. The headspace of each sample was briefly sparged with nitrogen gas, and the samples were flash frozen in liquid nitrogen. The samples were placed on the freeze dryer, and all thawed rapidly. It is likely that most of the drying was achieved by solvent evaporation rather than freeze drying; even if some material was dried before the samples thawed, it may have been re-dissolved in the remaining liquid. The occurrence or extent of freeze drying cannot be confirmed or measured. After drying, the samples containing HPMCP and Poloxamers 188 and 407 appeared to be powdery, while the samples containing HPMC, HPMCAS, and Povidone K-90 appeared as glassy films coating the inner surfaces of the vials. Complete drying of the samples required two to three days.

TABLE 9
Additives and solvents for solid dispersions of Method 1.

| Solid | | | |
|---|---|---|---|
| dis- | | | |
| persion | | | Total |
| # | Additive | Solvent(s) | volume |
| 26 | HPMC¹ | 6:1 DMF:water | 35 mL |
| 27 | HPMCAS¹ | 14:7:2:1 Acetone:methanol:DMF:water | 120 mL |
| 28 | HPMCP | 4:1:1 Methanol:water:acetone | 30 mL |
| 29 | Poloxamer | 8:1 Ethanol:water | 45 mL |
| | 188 | | |
| 30 | Poloxamer | 8:1 Ethanol:water | 45 mL |
| | 407 | | |
| 31 | Povidone | Ethanol | 20 mL |
| | K-90 | | |

¹Did not dissolve completely; centrifuged and dried supernatant.

### Preparation of Solid Dispersions—Method 2 Flash Freezing and Solvent Evaporation by Centrifugal Concentrator

Approximately 100 mg of compound (1) and 400 mg of the specified additive (Table 10) were dispensed into each of five tubes. The mixtures were dissolved in the solvents systems described in Table 10. Aliquots of 1 mL of each solution were distributed into 2 mL microcentrifuge tubes. The samples were flash frozen in liquid nitrogen, allowing the material to incubate for approximately five minutes to equilibrate to a lower temperature. The samples were opened and placed into the centrifugal concentrator at ambient temperature. Centrifugation and evacuation were initiated immediately. The samples did not remain frozen; however, the samples containing acetone dried by solvent evaporation in approximately two hours, and those containing ethanol dried after an additional 30 minutes at 60° C. The samples containing poloxamer were powdery, while the samples containing HPMCAS, HPMCP, and Povidone were glassy and brittle.

TABLE 10
Additives and solvents for solid dispersions of Method 2.

| Solid dispersion # | Additive | Solvent | Volume |
|---|---|---|---|
| 32 | HPMCAS¹ | Acetone | 25 mL |
| 33 | HPMCP | Acetone | 25 mL |
| 34 | Poloxamer 188 | Acetone | 20 mL |
| 35 | Poloxamer 407 | Acetone | 20 mL |
| 36 | Povidone K-90 | Ethanol | 20 mL |

¹Did not dissolve completely; centrifuged and dried supernatant.

### In Vitro Assessment

The performance of the solid dispersion formulations was evaluated in vitro by dilution into fasted- and fed-state simulated gastric and intestinal fluids.

For the fasted-state experiment, a quantity of approximately 50 mg of sample was dispensed into a microcentrifuge tube, and a volume of approximately 500 μL of FaSSGF was added (a 1:10 (w/v) dilution). The samples were briefly mixed by vortex agitation and placed on a rotator at ambient temperature for 15 minutes. The samples were again mixed by vortex agitation, and a 50 μL aliquot of each suspension was diluted into 500 μL FaSSIF. These samples were briefly mixed by vortex agitation and placed on the rotator for 15 minutes at ambient temperature. The pH values of the SGF and SIF samples were measured.

The fed-state experiment was performed in the same way, except a volume of approximately 500 μL of FeSSGF was added to a quantity of approximately 25 mg solid (a 1:20 (w/v) dilution). The FeSSGF sample was diluted into FeSSIF as described above.

Upon completion of each dilution experiment (incubation in simulated fluids for 15 minutes with agitation), the samples were centrifuged and a portion of the clear solution was diluted as necessary and analyzed by HPLC.

The results of the in vitro evaluation of the formulations are presented in Table 11. The recovery in SGF and SIF from each formulation was determined from the theoretical concentration, in mg/mL, in each SGF sample based on the mass of the solid, the percentage of compound (1) in that formulation, and the volume of medium added.

TABLE 11
In vitro evaluation of solid dispersions.

| | | [Compound (1)] | % Recovery | |
|---|---|---|---|---|
| | | in medium, | from | |
| Solid dispersion # | Medium | mg/mL | theoretical | pH |
| 26 | FaSSGF¹ | (0.374) | (1.8%) | 1.80 |
| | FaSSIF^{1,2} | (0.210) | Not calculated | 6.27 |
| | FeSSGF¹ | (2.43) | (23.2%) | 5.04 |
| | FeSSIF² | 0.060 | Not calculated | 5.69 |
| 27 | FaSSGF | 0.281 | 1.4% | 1.88 |
| | FaSSIF² | 0.029 | Not calculated | 6.14 |
| | FeSSGF³ | <0.004 | <0.04% | 5.01 |
| | FeSSIF² | 0.062 | Not calculated | 5.60 |
| 28 | FaSSGF | 0.235 | 1.1% | 1.86 |
| | FaSSIF | 0.056 | 3.0% | 6.12 |
| | FeSSGF³ | <0.004 | <0.04% | 4.68 |
| | FeSSIF | 0.087 | 9.3% | 5.63 |
| 29 | FaSSGF | 0.274 | 1.4% | 1.79 |
| | FaSSIF³ | <0.004 | <0.2% | 6.44 |
| | FeSSGF | 0.012 | 0.1% | 5.06 |
| | FeSSIF | 0.051 | 5.6% | 5.69 |
| 30 | FaSSGF | 0.449 | 2.2% | 1.82 |
| | FaSSIF | 0.009 | 0.5% | 6.43 |
| | FeSSGF | 0.055 | 0.5% | 5.05 |
| | FeSSIF | 0.068 | 7.2% | 5.70 |
| 31 | FaSSGF | 0.153 | 0.7% | 1.91 |
| | FaSSIF³ | <0.004 | <0.2% | 6.46 |
| | FeSSGF¹ | (0.047) | (0.5)% | 5.02 |
| | FeSSIF | 0.061 | 6.5% | 5.68 |
| 32 | FaSSGF | 0.212 | 1.1% | 1.84 |
| | FaSSIF² | 0.030 | Not calculated | 5.96 |
| | FeSSGF³ | <0.004 | <0.04% | 5.01 |
| | FeSSIF² | 0.072 | Not calculated | 5.55 |
| 33 | FaSSGF | 0.038 | 0.2% | 1.77 |
| | FaSSIF² | 0.058 | Not calculated | 5.66 |
| | FeSSGF³ | <0.004 | <0.04% | 4.97 |
| | FeSSIF² | 0.312 | Not calculated | 5.39 |
| 34 | FaSSGF | 0.246 | 1.3% | 1.79 |
| | FaSSIF | 0.005 | 0.3% | 6.44 |
| | FeSSGF³ | <0.004 | <0.04% | 5.04 |
| | FeSSIF | 0.042 | 4.8% | 5.69 |
| 35 | FaSSGF | 0.344 | 1.8% | 1.80 |
| | FaSSIF | 0.007 | 0.4% | 6.42 |
| | FeSSGF | 0.064 | 0.7% | 5.05 |
| | FeSSIF | 0.041 | 4.6% | 5.70 |
| 36 | FaSSGF | 0.162 | 0.9% | 1.93 |
| | FaSSIF | 0.013 | 0.8% | 6.44 |
| | FeSSGF¹ | (0.398) | (4.3%) | 5.06 |
| | FeSSIF | 0.075 | 8.9% | 5.70 |

¹Could not remove clear aliquot of supernatant - concentration (in parentheses) is higher than actual value
²Added solid from SGF sample to SIF - could not calculate recovery based on volumetric dilution factor
³Concentration of HPLC sample below LOQ. Percent recovery is therefore presented as less than the recovery of a sample with a concentration at the LOQ.

### Example 21

### Physical Mixtures

### Preparation of Physical Mixtures

Approximately 100 mg of compound (1) was dispensed into a mortar. A quantity of approximately 400 mg of the appropriate additive (Table 12) was added. Each mixture was ground with a pestle until it appeared uniform by visual observation. The poloxamers were ground into fine powders, while the other additives, particularly HPMCAS and HPMCP, maintained larger and more varied particles.

TABLE 12
Additives for physical mixtures.

| Physical mixture # | Additive |
|---|---|
| 37 | HPMC |
| 38 | HPMCAS |
| 39 | HPMCP |
| 40 | Poloxamer 188 |
| 41 | Poloxamer 407 |
| 42 | Povidone K-90 |

### In Vitro Assessment

The performance of the physical mixtures was evaluated in vitro by dilution into fasted- and fed-state simulated gastric and intestinal fluids.

For the fasted-state experiment, a quantity of approximately 50 mg of sample was dispensed into a microcentrifuge tube, and a volume of approximately 500 μL of FaSSGF was added (a 1:10 (w/v) dilution). The samples were briefly mixed by vortex agitation and placed on a rotator at ambient temperature for 15 minutes. The samples were again mixed by vortex agitation, and a 50 μL aliquot of each suspension was diluted into 500 μL FaSSIF. These samples were briefly mixed by vortex agitation and placed on the rotator for 15 minutes at ambient temperature. The pH values of the SGF and SIF samples were measured.

The fed-state experiment was performed in the same way, except a volume of approximately 500 μL of FeSSGF was added to a quantity of approximately 25 mg solid (a 1:20 (w/v) dilution). The FeSSGF sample was diluted into FeSSIF as described above.

Upon completion of each dilution experiment (incubation in simulated fluids for 15 minutes with agitation), the samples were centrifuged and a portion of the clear solution was diluted as necessary and analyzed by HPLC.

The results of the in vitro evaluation of the formulations are presented in Table 11. The recovery in SGF and SIF from each formulation was determined from the theoretical concentration, in mg/mL, in each SGF sample based on the mass of the solid, the percentage of compound (1) in that formulation, and the volume of medium added.

TABLE 13
In vitro evaluation of physical mixtures.

| Physical | | [Compound (1)] in | % Recovery from | |
|---|---|---|---|---|
| mixture# | Medium | medium, mg/mL | theoretical | pH |
| 37 | FaSSGF¹ | (3.04) | (14.9%) | 1.80 |
| | FaSSIF² | <0.004 | <0.2% | 6.41 |
| | FeSSGF² | <0.004 | <0.04% | 5.01 |
| | FeSSIF | 0.034 | 3.7% | 5.69 |
| 38 | FaSSGF | 0.095 | 0.5% | 1.74 |
| | FaSSIF² | <0.004 | <0.2% | 6.34 |
| | FeSSGF² | <0.004 | <0.04% | 4.98 |
| | FeSSIF | 0.046 | 5.1% | 5.68 |
| 39 | FaSSGF | 0.144 | 0.7% | 1.75 |
| | FaSSIF² | <0.004 | <0.2% | 6.20 |
| | FeSSGF² | <0.004 | <0.04% | 4.57 |
| | FeSSIF | 0.048 | 5.3% | 5.63 |
| 40 | FaSSGF | 0.289 | 1.4% | 1.71 |
| | FaSSIF² | <0.004 | <0.2% | 6.44 |
| | FeSSGF | 0.009 | 0.1% | 5.04 |
| | FeSSIF | 0.049 | 5.3% | 5.69 |
| 41 | FaSSGF | 0.325 | 1.6% | 1.76 |
| | FaSSIF² | <0.004 | <0.2% | 6.43 |
| | FeSSGF | 0.039 | 0.4% | 5.03 |
| | FeSSIF | 0.053 | 5.9% | 5.69 |
| 42 | FaSSGF | 0.257 | 1.3% | 1.86 |
| | FaSSIF² | <0.004 | <0.2% | 6.43 |
| | FeSSGF¹ | (0.076) | (0.8%) | 5.01 |
| | FeSSIF | 0.048 | 5.4% | 5.65 |

¹Could not remove clear aliquot of supernatant - concentration (in parentheses) is higher than actual value
²Concentration of HPLC sample below LOQ. Percent recovery is therefore presented as less than the recovery of a sample with a concentration at the LOQ.

Preparation of Spray-Dried Solid Dispersions

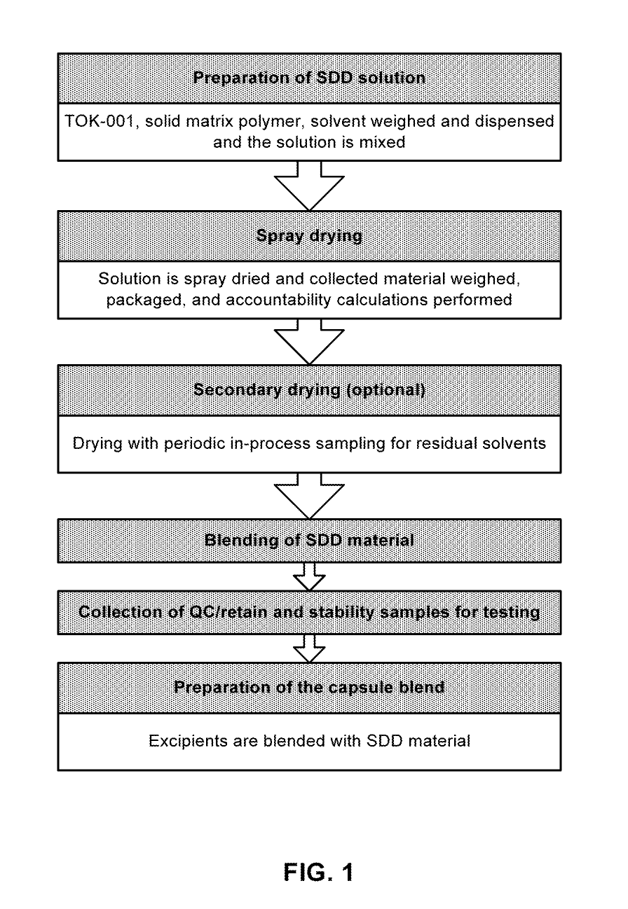

FIG. 1 depicts a general workflow for preparing a spray dried dispersion formulation of TOK-001. Two processes are employed in the manufacture of the final TOK-001 drug product. In the first process, spray dried dispersions (SDDs) of the TOK-001 are prepared by mixing TOK-001 with a solid matrix polymer (e.g., HPMCAS) in a solvent, followed by spray-drying the mixture to form a spray dried dispersion (SDD) drug product intermediate (DPI). Optionally, the SDD DPI undergoes a secondary drying process to remove residual solvent. In the second process, SDD DPI is blended with encapsulation excipients and filled in capsules.

SDD Feed Solution Development

The solubility of the API was assessed in several common solvents systems to ensure obtaining the maximum API concentration for the spray drying process. Based on the data shown in Table 14 below, the solvent system 2:1, methanol:THF showed the highest API solubility and was originally retained as the binary system of choice for the spray drying process development. Subsequently this binary solvent mixture was changed to the 2:1, methanol:acetone mixture due to long secondary drying times observed with the 2:1, methanol:THF system.

TABLE 14
API Solubility Assessment in Common Solvents

| Solvent System | Amount added (g) | API Solubility (%) |
|---|---|---|
| Methanol | 1.04 | 4.16 |
| Ethanol | 1.17 | 4.68 |
| Ethyl Acetate | 0.13 | 0 |
| Acetone | 0.26 | 1.04 |
| Tetrahydrofuran | 1.82 | 7.28 |
| 2:1 Acetone:Methanol | 1.04 | 4.16 |
| 2:1 Methanol:Tetrahydrofuran | 4.16 | 16.64 |
| 2:1 Methanol:Acetone | 1.82 | 7.28 |

Spray Drying Parameters

The optimum parameters used for the spray drying process for the 2:1, methanol:acetone with API (5.0%) and HPMCAS solution were based on the manufacturer's experience with the solvents and polymer used in this SDD application.

To optimize the spray-drying process, various parameters of the spray drying process were tested and the resulting SDD particles assessed for bulk density, tapped density, mean particle size, and particle size distribution. Results from TOK-001: HPMCAS SDD samples are summarized in Table 15.

TABLE 15
SDD optimization parameters

| | | | | | | | Mean |
|---|---|---|---|---|---|---|---|
| | Inlet | Outlet | Atomization | | Bulk | Tapped | Particle |
| | Temp | Temp | pressure | Feed | Density | Density | Size |
| Run | (° C.) | (° C.) | (bar) | Rate | (g/ml) | (g/ml) | (μm) |
| 1 | 90 | 65 | 1.2 | 25 | 0.346 | 0.477 | 14 |
| 2 | 110 | 65 | 1.2 | 45 | 0.281 | 0.409 | 11 |
| 3 | 100 | 60 | 1 | 40 | 0.33 | 0.441 | 14 |
| 4 | 110 | 55 | 1.2 | 55 | 0.304 | 0.427 | 15 |
| 5 | 110 | 55 | 0.8 | 55 | 0.284 | 0.399 | 20 |
| 6 | 110 | 65 | 0.8 | 45 | 0.275 | 0.4 | 21 |
| 7 | 90 | 55 | 0.8 | 35 | 0.313 | 0.439 | 15 |
| 8 | 100 | 60 | 1 | 40 | 0.298 | 0.433 | 12 |
| 9 | 100 | 60 | 1 | 40 | 0.307 | 0.431 | 11 |
| 10 | 90 | 55 | 1.2 | 35 | 0.327 | 0.459 | 12 |
| 11 | 90 | 65 | 0.8 | 25 | 0.322 | 0.444 | 12 |

Based on results from the optimization studies, the following spray drying parameters were adopted for the TOK-001 HPMCAS SDD DPI manufacturing step as shown below in Table 16.

TABLE 16
SDD Parameters Used for the HPMCAS Formulation Process

| | | Atomization | Process |
|---|---|---|---|
| Inlet Temp | Outlet Temp | Pressure | Gas Flow |
| 120° C. | 65° C. | 1.0 bar | 80 kg/hr (75-90 kg/hr) |
| (10° C.-140° C.) | (50° C.-75° C.) | (0.8-1.2 bar) | |

Secondary Drying

Following the spray-drying process, samples were vacuum dried in an oven at 50° C. and −25 in Hg. Samples were routinely taken intermittently during the secondary drying process of SDD lots or sublots and analyzed by gas chromatography for residual solvents (methanol and acetone). The drying process target value is <4000 ppm for acetone (ICH limit is 5000 ppm) and <2000 ppm for methanol (ICH limit is 3000 ppm).

Following the drying process, TOK-001 SDD particles were further blended with excipients, then compacted into capsules or tablets. Table 17 depicts one exemplary capsule formulation for the TOK-001:HPMCAS SDD.

TABLE 17
Formulation for TOK-001 HPMCAS SDD Capsule

| Granulation Components | Item No | mg/capsule |
|---|---|---|
| 50/50 TOK-001/HPMCAS-LG Spray Dried | PN460 | 100 |
| Dispersion | | |
| Lactose Monohydrate, NF (Tablettose 80) | E0031 | 58 |
| Crospovidone, NF (Polyplasdone XL) | E0115 | 30 |
| Poloxamer 188, NF (Kolliphor P188) | E0063 | 10 |
| Colloidal Silicon Dioxide, NF (Cabosil M-5P) | E0021 | 1 |
| Magnesium Stearate, NF (Hyqual 5712) | E0020 | 1 |
| Capsule Fill Weight: | | 200 |

Table 18 depicts another exemplary capsule formulation for the TOK-001:HPMCAS SDD.

TABLE 18
Capsule formulation of TOK-001: HPMCAS SDD

| | | |
|---|---|---|
| 50/50 TOK-001/HPMCAS-LG Spray | 78.4 | 1000 |
| Dried Dispersion | | |
| Dicalcium Phosphate, NF | 9.4 | 120 |
| (DiCafos) | | |
| Disintegrant | 3.1 | 39 |
| (CCS) | | |
| Poloxamer 188, NF | 7.8 | 100 |
| (Pluronic F-68) | | |
| Colloidal Silicon Dioxide, NF | 0.6 | 4.4 |
| (Cabosil M-5P) | | (Intra/Extra) |
| Magnesium Stearate, NF | 0.6 | 4.4 |
| (Hyqual 5712) | | (Intra/Extra) |
| Blend Total | 100.0 | 1275 |

Table 19 depicts an exemplary capsule formulation of a 1:1 TOK-001: copovidone SDD composition.

TABLE 19
capsule formulation of TOK-001:copovidone SDD.

| | Percent weight of |
|---|---|
| Component | composition |
| 50/50 TOK-001/Copovidone Spray Dried Dispersion | 50 |
| microcrystalline cellulose | 29 |
| Crospovidone, NF (Polyplasdone XL) | 15 |
| Hypromellose | 5 |
| Colloidal Silicon Dioxide, NF (Cabosil M-5P) | 0.5 |
| Magnesium Stearate, NF (Hyqual 5712) | 0.5 |
| Total: | 100 |

Amorphous Stability Data.

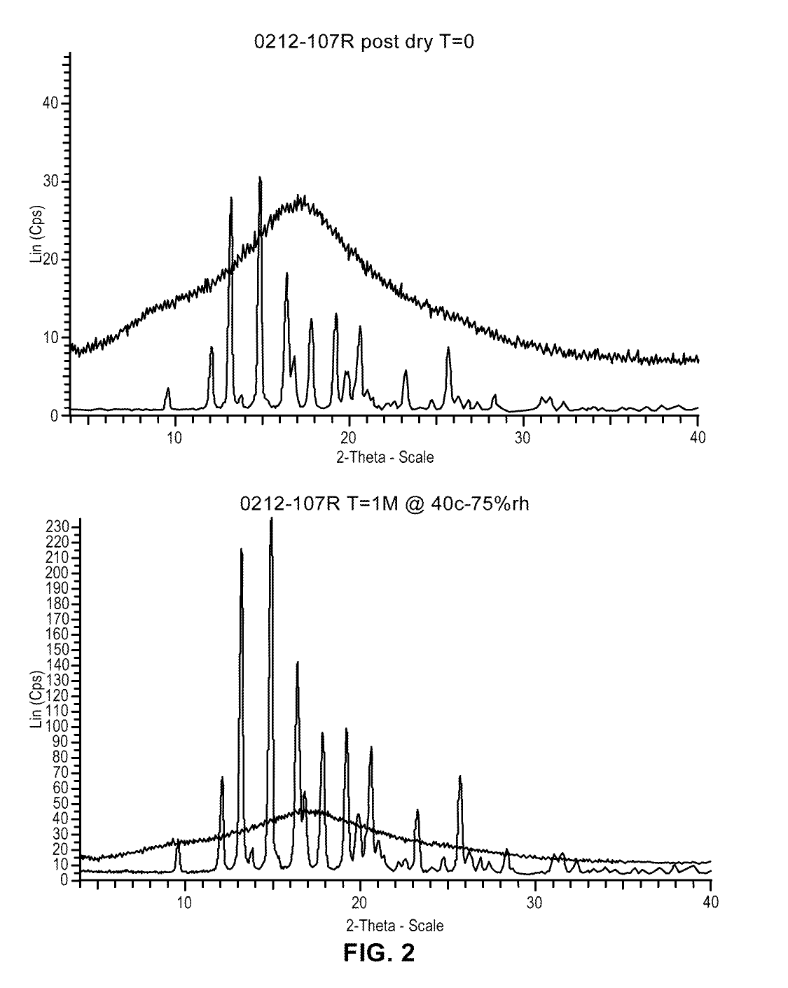

X-ray diffraction was performed on a Bruker D8 Focus, using a copper tube element and a PSD: LynxEye detector. The following data acquisition parameters were used: Volts: 40 kV, Power: 40 mA, Scan Range: 4.0000°-39.9960° 2θ, Number of Steps: 1685, Time/step: 0.3 s, Collection Time: 549s, Rotation Speed: 15 rpm, Mode: Continuous. FIG. 2A depicts an XRPD plot of TOK-001:HPMCAS-SDD particles at T=0 after spray-drying, vs. micronized crystalline TOK-001, demonstrating that the HPMCAS-SDD composition is highly amorphous. FIG. 2B depicts an XRPD plot of TOK-001:HPMCAS-SDD vs. micronized crystalline TOK-001 after storage for one month at 40° C./75% RH, demonstrating that the HPMCAS-SDD composition remains highly amorphous for at least one month without reverting back to crystalline form.

Pharmaceutical Formulation Testing

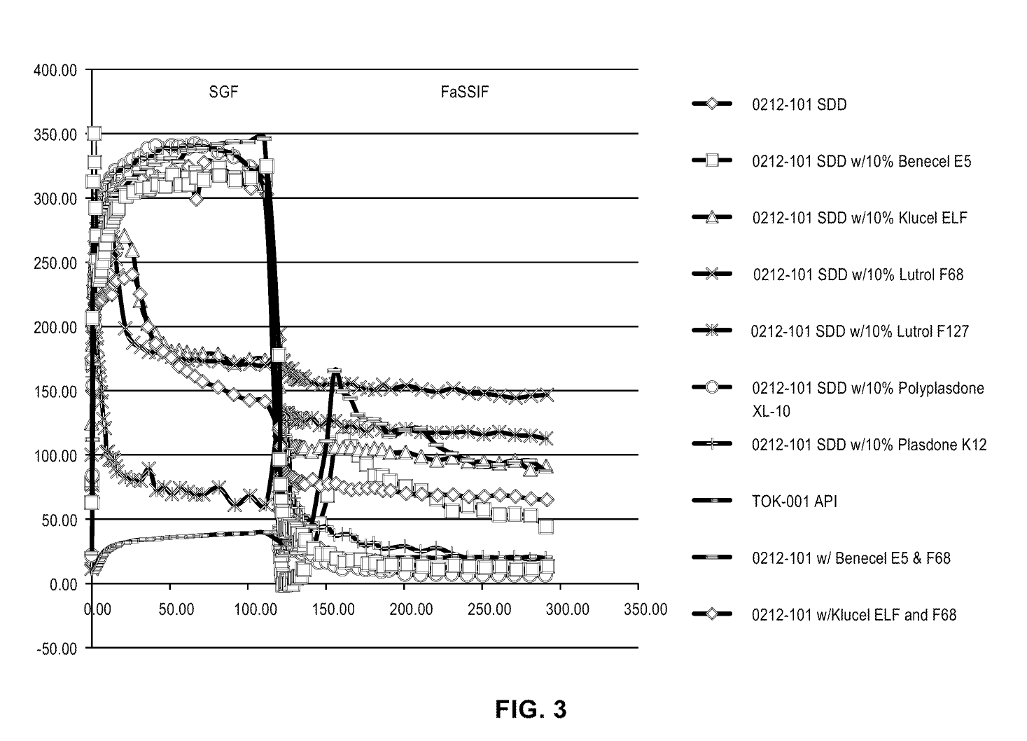

A panel of recrystallization inhibitors were tested in the TOK-001:HPMCAS formulations. The impact of various recrystallization inhibitors on solubility of the TOK-001: HPMCAS SDD compositions in SGF and after transition from SGF to FaSSIF is depicted in FIG. 3. Results indicate that poloxamer 188 (Lutrol F68) greatly reduces the difference in solubility between SGF conditions and after transition from SGF to FaSSIF.

Disintegration/Dispersability

Following compaction into capsules using standard methods, disintegration/dispersability of the capsules in 0.1N HCl and IAW USP 701 were tested. Results are depicted in Tables 20-21.

TABLE 20
TOK-001 HPMCAS capsules, T = 0

| | Disintegration | |
|---|---|---|
| Lot# | time (min) | Dispersibility Observation |
| 0212-232A | 2-4 | After capsule shell dissolved, material |
| | | dispersed, complete disintegration </= 4 |
| | | min. |
| 0212-232B | 2-4 | After capsule shell dissolved, material |
| | | dispersed, complete disintegration </= 4 |
| | | min. |
| 0212-233A | 2-3 | After capsule shell dissolved, material |
| | | dispersed, complete disintegration </= 3 |
| | | min. |
| 0212-233B | 2-3 | After capsule shell dissolved, material |
| | | dispersed, complete disintegration </= 3 |
| | | min. |

TABLE 21
TOK-001 HPMCAS capsules after storage at 40° C./75% RH.

| | Disintegration | |
|---|---|---|
| Lot# | time (min) | Dispersibility Observation |
| 0212-232A | 3-5 | After capsule shell dissolved, material |
| | | dispersed, complete disintegration </= 5 |
| | | min. |
| 0212-232B | 2-4 | After capsule shell dissolved, material |
| | | dispersed, complete disintegration </= 4 |
| | | min. |
| 0212-233A | 4 | After capsule shell dissolved, material |
| | | dispersed, complete disintegration </= 4 |
| | | min. |
| 0212-233B | 3-4 | After capsule shell dissolved, material |
| | | dispersed, complete disintegration </= 4 |
| | | min. |

These capsule formulations exhibit excellent disintegration at least up to two week after storage at high temperature and humidity.

Dissolution Testing

Dissolution behavior and solubility enhancement was tested using the μDISS Profiler™ (Pion Inc.), a small-volume, 8-shannel in situ UV dissolution apparatus, to collect concentration-time profiles in biorelevant media (e.g., FaSSIF). 3 capsule formulations were tested: 325 mg TOK-001 as micronized PIC, 50 mg TOK-001 HPMCAS SDD, 50 mg TOK-001 copovidone SDD. Dissolution testing proceeded according to the protocol in Table 22.

TABLE 22
Dissolution testing

Apparatus
USP Type-2
Dissolution Media
FaSSIF

| Sampling Time | Paddle Speed | Temperature |
|---|---|---|
| 5 Minutes | 75 RPM | 37° C. |
| 15 Minutes | 75 RPM | 37° C. |
| 30 Minutes | 75 RPM | 37° C. |
| 45 Minutes | 75 RPM | 37° C. |
| 60 Minutes | 75 RPM | 37° C. |

Note:
Samples are analyzed by HPLC-UV absorbance at 264 nm in comparison to a reference standard of TOK-001 dissolved in methanol at 20 mg/100 mL.

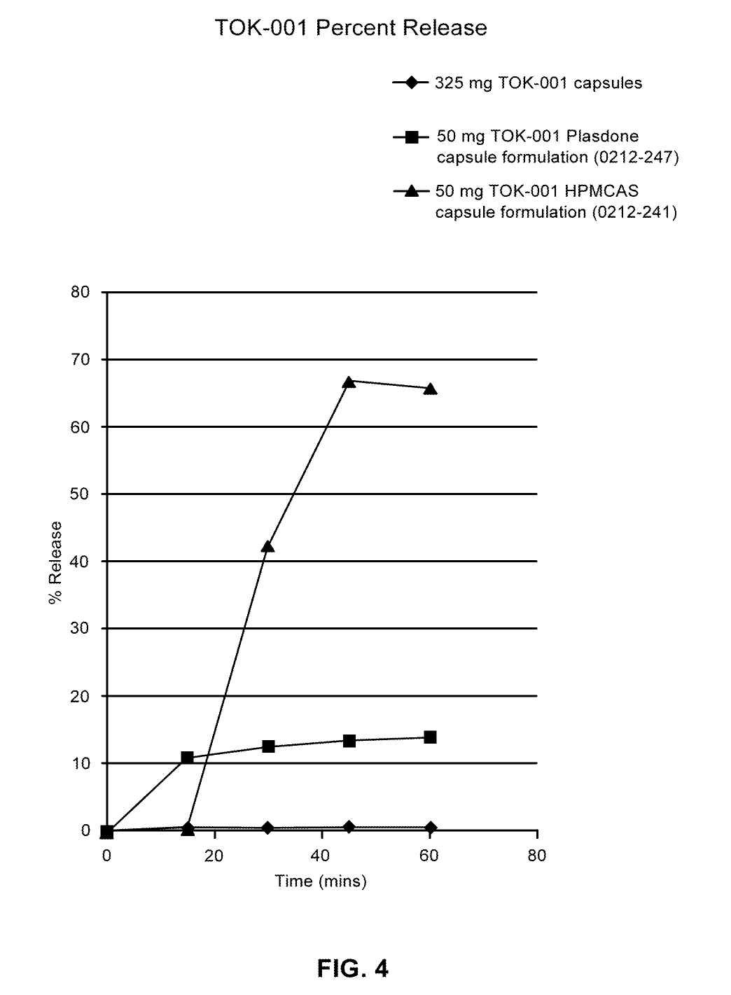

FIG. 4 depicts dissolution of the above three formulations as percent compound released into FaSSIF over time. The HPMCAS SDD formulation exhibits a 100 fold higher dissolution compared to the PIC formulation. By comparison, the copovidone SDD formulation exhibits a 15 fold higher dissolution rate compared to the PIC formulation.

Canine Pharmacokinetic Studies.

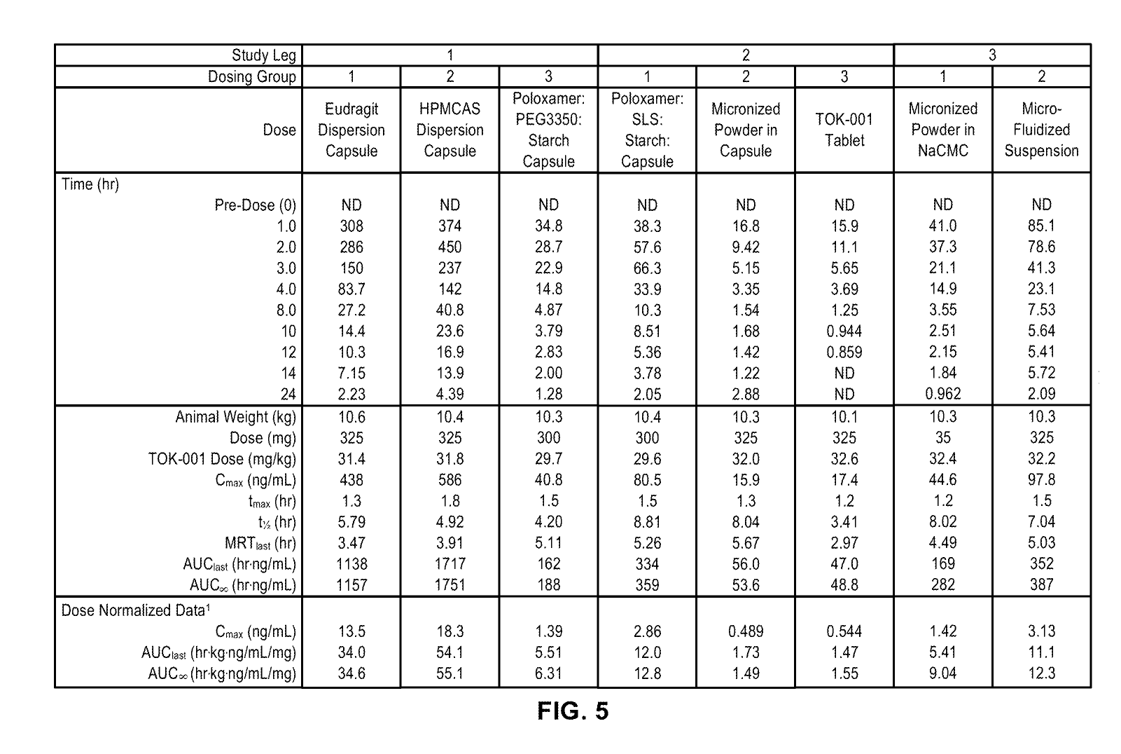

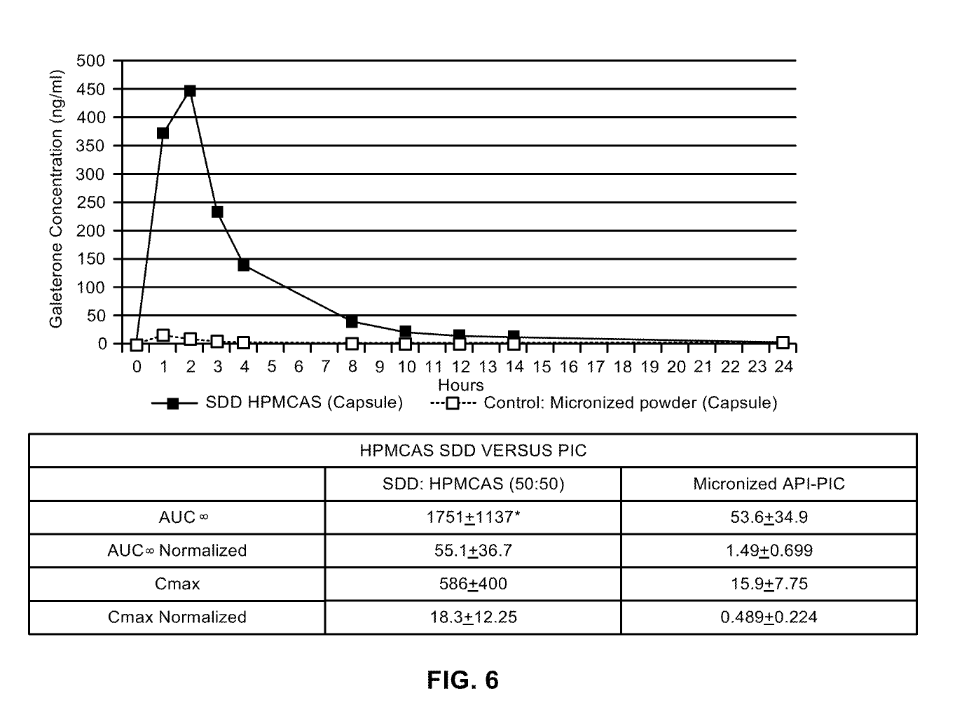

The oral exposure of TOK-001 in male Beagle dogs was evaluated after administration of various formulations of TOK-001. Subjects were fasted for 12 hours prior to administration. Blood samples were collected up to 24 hours post dose. Plasma concentrations were determined with a qualified LC-MS/MS method and pharmacokinetic parameters, summarized in FIG. 5, were determined for the TOK-001 plasma data. FIG. 6 depicts plasma concentrations of TOK-001 (referred to here as Galaterone) over time, comparing the TOK-001:HPMCAS SDD capsule formulation to the micronized crystalline PIC capsule formulation.

Fed/Fasted Pharmacokinetics

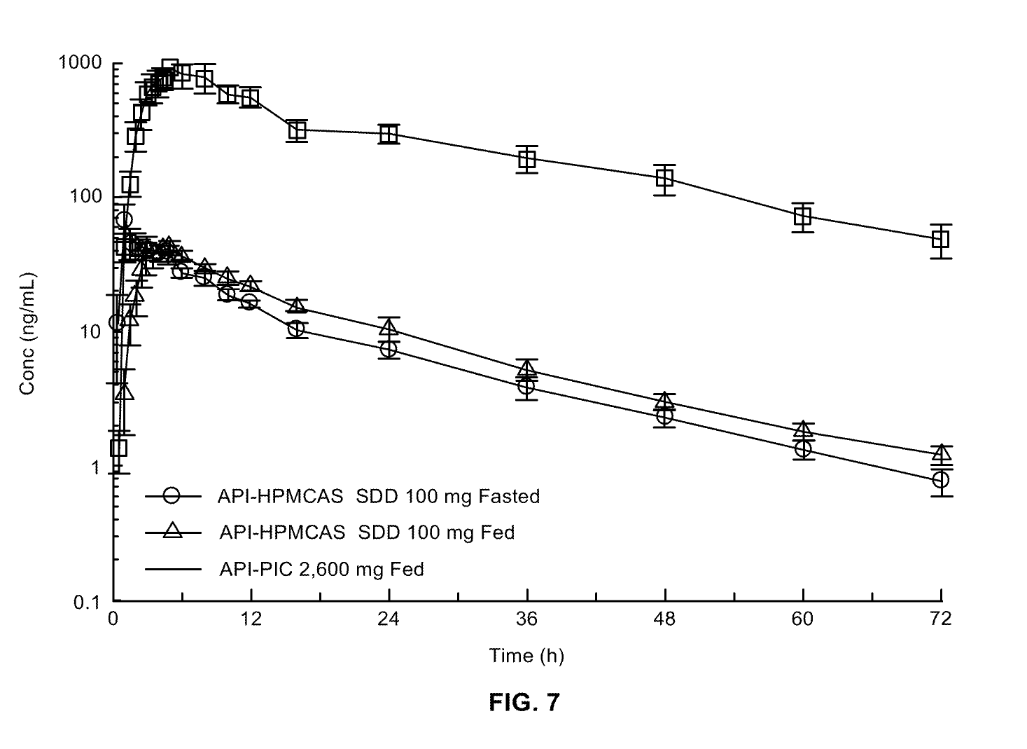

To test the pharmacokinetics of the HPMCAS SDD formulation, a human crossover trial was conducted in which human subjects were orally administered (1) HPMCAS SDD capsule containing 100 mg TOK-001 while in a fed state, (2) HPMCAS SDD capsule containing 100 mg TOK-001 while in a fasted state, and 2600 mg TOK-001 micronized powder in capsule (PIC) while in a fed state. Blood samples were taken at regular time points following each administration for up to 72 hours, and plasma concentrations of TOK-001 assessed. FIG. 7 depicts TOK-001 plasma concentrations over time for HPMCAS SDD-Fed, HPMCAS SDD-Fasted, and PIC-Fed. Subjects administered HPMCAS SDD in a fed state exhibit similar plasma concentration profiles as when administered HPMCAS SDD in a fasted state.

Table 23 depicts a summary of pharmacokinetic data from oral administration of HPMCAS SDD-Fed, HPMCAS SDD-Fasted, and PIC-fed conditions. The summary demonstrates a ˜16% increase in AUC_{(inf)} but a ˜22% decrease in Cₘₐₓ when the HPMCAS-SDD formulation is given with food. By contrast, there is a ˜13-fold increase in AUC when the PIC formulation is given with food compared to PIC given in a fasted state.

TABLE 23
Summary of pharmacokinetic parameters for TOK-001 after oral administration
of API-HPMCAS SDD 100 mg fasted and fed and API-PIC 2,600 mg fed to
healthy subjects. Data represents mean +/− SD of n = 6 subjects.

| | API-HPMCAS SDD | API-HPMCAS SDD | API-PIC |
|---|---|---|---|
| Parameter* | 100 mg Fasted | 100 mg Fed | 2,600 mg Fed |
| Cmax (ng/mL) | 69.3 ± 46.9 (6) | 47.2 ± 13.2 (6) | 1,153 ± 458 (6) |
| Tmax(h) | 1.00 (6) | 4.50 (6) | 5.50 (6) |
| | [1.00-5.03] | [3.00-12.0] | [4.07-8.00] |
| AUC(0-t) (h × ng/mL) | 617 ± 204 (6) | 694 ± 134 (6) | 18,165 ± 6,235 (6) |
| AUC(inf) (h × ng/mL) | 638 ± 207 (6) | 721 ± 143 (6) | 19,306 ± 6,919 (6) |
| λz (1/h) | 0.0451 ± 0.0077 (6) | 0.0446 ± 0.0043 (6) | 0.0449 ± 0.0055 (6) |
| t½ (h) | 15.7 ± 2.45 (6) | 15.7 ± 1.46 (6) | 15.6 ± 1.94 (6) |
| CL/F (mL/min) | 2,869 ± 986 (6) | 2,396 ± 521 (6) | 2,641 ± 1,409 (6) |
| Vz/F (L) | 3,943 ± 1,689 (6) | 3,241 ± 706 (6) | 3,579 ± 2,042 (6) |

*Arithmetic mean ± standard deviation (N) except Tmax for which the median (N) [Range] is reported.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A solid dispersion pharmaceutical composition comprising
a) a compound of Formula I: 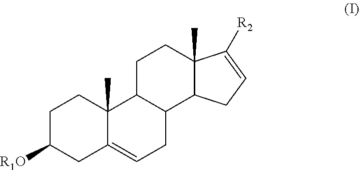
or a pharmaceutically acceptable salt, N-oxide, active metabolite, prodrug, or solvate thereof;
wherein:
R₁ is H or acetyl;
R₂ is pyridyl or benzimidazolyl; and
b) a solid matrix;
c) wherein said compound is dispersed in said solid matrix.

2. The solid dispersion pharmaceutical composition of claim 1, wherein said solid matrix comprises a polymer.

3. The solid dispersion pharmaceutical composition of claim 2, wherein said polymer is soluble in an aqueous solution.

4. The solid dispersion pharmaceutical composition of claim 3, wherein said aqueous solution is water.

5. The solid dispersion pharmaceutical composition of claim 3, wherein said aqueous solution has a pH of 5.0 or greater.

6-83. (canceled)

84. The solid dispersion pharmaceutical composition of claim 1, wherein said compound is substantially in a non-crystalline form, and wherein the bioavailability of the compound when administered to a subject in a fasted state is substantially the same as the bioavailability of the drug when administered to said subject in a fed state.

85-96. (canceled)

97. The solid dispersion pharmaceutical composition of claim 1, wherein said compound is a compound of Formula II: 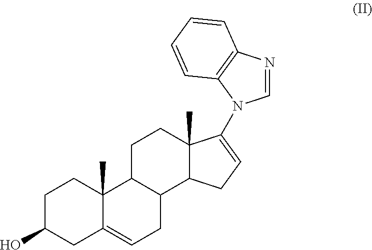 [SMILES: C[C@]12CC[C@H](O)CC1=CCC1C2CC[C@]2(C)C(n3cnc4ccccc43)=CCC12]
or a pharmaceutically acceptable salt, N-oxide, active metabolite, prodrug, or solvate thereof.

98-103. (canceled)

104. A method of making a solid dispersion composition comprising a compound of Formula I: 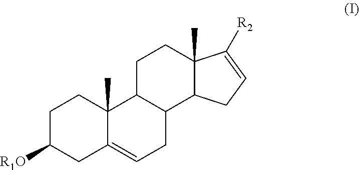
or a pharmaceutically acceptable salt, N-oxide, active metabolite, prodrug, or solvate thereof;
wherein:
R₁ is H or acetyl;
R₂ is pyridyl or benzimidazolyl; the method comprising the steps of:
a) forming a solution comprising said compound, said solid matrix, and a solvent; and
b) substantially removing said solvent, thereby resulting in the solid dispersion composition.

105-125. (canceled)

126. The method of claim 104, wherein said compound is a compound of Formula II: 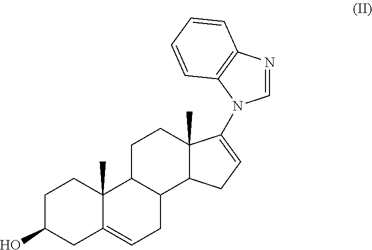 [SMILES: C[C@]12CC[C@H](O)CC1=CCC1C2CC[C@]2(C)C(n3cnc4ccccc43)=CCC12]
or a pharmaceutically acceptable salt, N-oxide, active metabolite, prodrug, or solvate thereof.

127-139. (canceled)

140. A method for treating prostate cancer in a subject in need thereof, comprising administering to said subject a solid dispersion pharmaceutical composition of claim 97.

141. (canceled)

142. The method of claim 140, wherein said prostate cancer is castration resistant prostate cancer.

143-149. (canceled)

150. The method of claim 140, wherein said composition is administered in multiple unit doses.

151-166. (canceled)

167. The method of claim 150, wherein the unit dose is a solid dosage form.

168-169. (canceled)

170. The method of claim 167, wherein the solid dosage form is a tablet.

171-189. (canceled)

190. The solid dispersion pharmaceutical composition of claim 1, wherein the composition is formulated for administration once a day.

191-204. (canceled)

205. The solid dispersion pharmaceutical composition of claim 97, which is a spray dried dispersion composition.

206. The method of claim 126, wherein the substantially removing comprises spray-drying the solution.

207. The method of claim 140, wherein the solid dispersion pharmaceutical composition is a spray dried dispersion composition.
